SEQUENCE LISTING (280 sequences)

SEQ ID NO: 1 - DNA, 12 bp, Artificial Sequence
  Comment for artificial sequence chemically pretreated Genom DNA
ttaataatcgat

SEQ ID NO: 2 - DNA, 12 bp, Artificial Sequence
  Comment for artificial sequence chemically pretreated Genom DNA
atcgattattaa

SEQ ID NO: 3 - DNA, 12 bp, Artificial Sequence
  Comment for artificial sequence chemically pretreated Genom DNA
atcgattattgg

SEQ ID NO: 4 - DNA, 12 bp, Artificial Sequence
  Comment for artificial sequence chemically pretreated Genom DNA
ccaataatcgat

SEQ ID NO: 5 - DNA, 11 bp, Artificial Sequence
  Comment for artificial sequence chemically pretreated Genom DNA
gtgtaatattt

SEQ ID NO: 6 - DNA, 11 bp, Artificial Sequence
  Comment for artificial sequence chemically pretreated Genom DNA
aaatattacac

SEQ ID NO: 7 - DNA, 11 bp, Artificial Sequence
  Comment for artificial sequence chemically pretreated Genom DNA
gggtattgtat

SEQ ID NO: 8 - DNA, 11 bp, Artificial Sequence
  Comment for artificial sequence chemically pretreated Genom DNA
atacaataccc

SEQ ID NO: 9 - DNA, 11 bp, Artificial Sequence
  Comment for artificial sequence chemically pretreated Genom DNA
gtgtaattttt

SEQ ID NO: 10 - DNA, 11 bp, Artificial Sequence
  Comment for artificial sequence chemically pretreated Genom DNA
aaaaattacac

SEQ ID NO: 11 - DNA, 16 bp, Artificial Sequence
  Comment for artificial sequence chemically pretreated Genom DNA
ctctaaaaataacccc

SEQ ID NO: 12 - DNA, 18 bp, Artificial Sequence
  Comment for artificial sequence chemically pretreated Genom DNA
tgttattaaaaatagaaa

SEQ ID NO: 13 - DNA, 18 bp, Artificial Sequence
  Comment for artificial sequence chemically pretreated Genom DNA
tttctatttttaataaca

SEQ ID NO: 14 - DNA, 18 bp, Artificial Sequence
  Comment for artificial sequence chemically pretreated Genom DNA
tttttatttttagtaata

SEQ ID NO: 15 - DNA, 18 bp, Artificial Sequence
  Comment for artificial sequence chemically pretreated Genom DNA
tattactaaaaataaaaa

SEQ ID NO: 16 - DNA, 18 bp, Artificial Sequence
  Comment for artificial sequence chemically pretreated Genom DNA
tgttattaaaaatagaat

SEQ ID NO: 17 - DNA, 18 bp, Artificial Sequence
  Comment for artificial sequence chemically pretreated Genom DNA
attctatttttaataaca

SEQ ID NO: 18 - DNA, 18 bp, Artificial Sequence
  Comment for artificial sequence chemically pretreated Genom DNA
gttttatttttagtaata

SEQ ID NO: 19 - DNA, 18 bp, Artificial Sequence
  Comment for artificial sequence chemically pretreated Genom DNA
tattactaaaaataaaac

SEQ ID NO: 20 - DNA, 11 bp, Artificial Sequence
  Comment for artificial sequence chemically pretreated Genom DNA
tgttgagttat

SEQ ID NO: 21 - DNA, 11 bp, Artificial Sequence
  Comment for artificial sequence chemically pretreated Genom DNA
ggggattgtat

SEQ ID NO: 22 - DNA, 11 bp, Artificial Sequence
  Comment for artificial sequence chemically pretreated Genom DNA
ataactcaaca

SEQ ID NO: 23 - DNA, 11 bp, Artificial Sequence
  Comment for artificial sequence chemically pretreated Genom DNA
atgatttagta

SEQ ID NO: 24 - DNA, 11 bp, Artificial Sequence
  Comment for artificial sequence chemically pretreated Genom DNA
tactaaatcat

SEQ ID NO: 25 - DNA, 11 bp, Artificial Sequence
  Comment for artificial sequence chemically pretreated Genom DNA
tgttgatttat

SEQ ID NO: 26 - DNA, 11 bp, Artificial Sequence
  Comment for artificial sequence chemically pretreated Genom DNA
ataaatcaaca

SEQ ID NO: 27 - DNA, 11 bp, Artificial Sequence
  Comment for artificial sequence chemically pretreated Genom DNA
gtgagttagta

SEQ ID NO: 28 - DNA, 11 bp, Artificial Sequence
  Comment for artificial sequence chemically pretreated Genom DNA
tactaactcac

SEQ ID NO: 29 - DNA, 10 bp, Artificial Sequence
  Comment for artificial sequence chemically pretreated Genom DNA
ggggattttt

SEQ ID NO: 30 - DNA, 10 bp, Artificial Sequence
  Comment for artificial sequence chemically pretreated Genom DNA
aaaaatcccc

SEQ ID NO: 31 - DNA, 10 bp, Artificial Sequence
  Comment for artificial sequence chemically pretreated Genom DNA
gggaattttt

SEQ ID NO: 32 - DNA, 11 bp, Artificial Sequence
  Comment for artificial sequence chemically pretreated Genom DNA
atacaatcccc

SEQ ID NO: 33 - DNA, 10 bp, Artificial Sequence
  Comment for artificial sequence chemically pretreated Genom DNA
aaaaattccc

SEQ ID NO: 34 - DNA, 10 bp, Artificial Sequence
  Comment for artificial sequence chemically pretreated Genom DNA
ggaaattttt

SEQ ID NO: 35 - DNA, 10 bp, Artificial Sequence
  Comment for artificial sequence chemically pretreated Genom DNA
aaaaatttcc

SEQ ID NO: 36 - DNA, 10 bp, Artificial Sequence
  Comment for artificial sequence chemically pretreated Genom DNA
gggatttttt

SEQ ID NO: 37 - DNA, 10 bp, Artificial Sequence
  Comment for artificial sequence chemically pretreated Genom DNA
aaaaaatccc

SEQ ID NO: 38 - DNA, 10 bp, Artificial Sequence
  Comment for artificial sequence chemically pretreated Genom DNA
ggaaagtttt

SEQ ID NO: 39 - DNA, 10 bp, Artificial Sequence
  Comment for artificial sequence chemically pretreated Genom DNA
aaaactttcc

SEQ ID NO: 40 - DNA, 10 bp, Artificial Sequence
  Comment for artificial sequence chemically pretreated Genom DNA
gggaagtttt

SEQ ID NO: 41 - DNA, 10 bp, Artificial Sequence
  Comment for artificial sequence chemically pretreated Genom DNA
aaaacttccc

SEQ ID NO: 42 - DNA, 11 bp, Artificial Sequence
  Comment for artificial sequence chemically pretreated Genom DNA
gggatttttta

SEQ ID NO: 43 - DNA, 11 bp, Artificial Sequence
  Comment for artificial sequence chemically pretreated Genom DNA
atgtaattttt

SEQ ID NO: 44 - DNA, 11 bp, Artificial Sequence
  Comment for artificial sequence chemically pretreated Genom DNA
taaaaaatccc

SEQ ID NO: 45 - DNA, 11 bp, Artificial Sequence
  Comment for artificial sequence chemically pretreated Genom DNA
tggaaagtttt

SEQ ID NO: 46 - DNA, 11 bp, Artificial Sequence
  Comment for artificial sequence chemically pretreated Genom DNA
aaaactttcca

SEQ ID NO: 47 - DNA, 21 bp, Artificial Sequence
  Comment for artificial sequence chemically pretreated Genom DNA
tttagtattacggatagaggt

SEQ ID NO: 48 - DNA, 21 bp, Artificial Sequence
  Comment for artificial sequence chemically pretreated Genom DNA
acctctatccgtaatactaaa

SEQ ID NO: 49 - DNA, 21 bp, Artificial Sequence
  Comment for artificial sequence chemically pretreated Genom DNA
gtttttgttcgtggtgttgaa

SEQ ID NO: 50 - DNA, 21 bp, Artificial Sequence
  Comment for artificial sequence chemically pretreated Genom DNA
ttcaacaccacgaacaaaaac

SEQ ID NO: 51 - DNA, 21 bp, Artificial Sequence
  Comment for artificial sequence chemically pretreated Genom DNA
tttagtattacggatagagtt

SEQ ID NO: 52 - DNA, 21 bp, Artificial Sequence
  Comment for artificial sequence chemically pretreated Genom DNA
aactctatccgtaatactaaa

SEQ ID NO: 53 - DNA, 21 bp, Artificial Sequence
  Comment for artificial sequence chemically pretreated Genom DNA
ggttttgttcgtggtgttgaa

SEQ ID NO: 54 - DNA, 11 bp, Artificial Sequence
  Comment for artificial sequence chemically pretreated Genom DNA
aaaaattacat

SEQ ID NO: 55 - DNA, 21 bp, Artificial Sequence
  Comment for artificial sequence chemically pretreated Genom DNA
ttcaacaccacgaacaaaacc

SEQ ID NO: 56 - DNA, 21 bp, Artificial Sequence
  Comment for artificial sequence chemically pretreated Genom DNA
tttagtattacggatagcgtt

SEQ ID NO: 57 - DNA, 21 bp, Artificial Sequence
  Comment for artificial sequence chemically pretreated Genom DNA
aacgctatccgtaatactaaa

SEQ ID NO: 58 - DNA, 21 bp, Artificial Sequence
  Comment for artificial sequence chemically pretreated Genom DNA
ggcgttgttcgtggtgttgaa

SEQ ID NO: 59 - DNA, 21 bp, Artificial Sequence
  Comment for artificial sequence chemically pretreated Genom DNA
ttcaacaccacgaacaacgcc

SEQ ID NO: 60 - DNA, 21 bp, Artificial Sequence
  Comment for artificial sequence chemically pretreated Genom DNA
tttagtattacggatagcggt

SEQ ID NO: 61 - DNA, 21 bp, Artificial Sequence
  Comment for artificial sequence chemically pretreated Genom DNA
accgctatccgtaatactaaa

SEQ ID NO: 62 - DNA, 21 bp, Artificial Sequence
  Comment for artificial sequence chemically pretreated Genom DNA
gtcgttgttcgtggtgttgaa

SEQ ID NO: 63 - DNA, 21 bp, Artificial Sequence
  Comment for artificial sequence chemically pretreated Genom DNA
ttcaacaccacgaacaacgac

SEQ ID NO: 64 - DNA, 10 bp, Artificial Sequence
  Comment for artificial sequence chemically pretreated Genom DNA
atatgtaaat

SEQ ID NO: 65 - DNA, 11 bp, Artificial Sequence
  Comment for artificial sequence chemically pretreated Genom DNA
atgtaatattt

SEQ ID NO: 66 - DNA, 10 bp, Artificial Sequence
  Comment for artificial sequence chemically pretreated Genom DNA
atttacatat

SEQ ID NO: 67 - DNA, 10 bp, Artificial Sequence
  Comment for artificial sequence chemically pretreated Genom DNA
atttgtatat

SEQ ID NO: 68 - DNA, 10 bp, Artificial Sequence
  Comment for artificial sequence chemically pretreated Genom DNA
atatacaaat

SEQ ID NO: 69 - DNA, 10 bp, Artificial Sequence
  Comment for artificial sequence chemically pretreated Genom DNA
ttatgtaaat

SEQ ID NO: 70 - DNA, 10 bp, Artificial Sequence
  Comment for artificial sequence chemically pretreated Genom DNA
atttacataa

SEQ ID NO: 71 - DNA, 10 bp, Artificial Sequence
  Comment for artificial sequence chemically pretreated Genom DNA
atttgtataa

SEQ ID NO: 72 - DNA, 10 bp, Artificial Sequence
  Comment for artificial sequence chemically pretreated Genom DNA
ttatacaaat

SEQ ID NO: 73 - DNA, 11 bp, Artificial Sequence
  Comment for artificial sequence chemically pretreated Genom DNA
atttgtatatt

SEQ ID NO: 74 - DNA, 11 bp, Artificial Sequence
  Comment for artificial sequence chemically pretreated Genom DNA
aatatacaaat

SEQ ID NO: 75 - DNA, 11 bp, Artificial Sequence
  Comment for artificial sequence chemically pretreated Genom DNA
ggtatgtaaat

SEQ ID NO: 76 - DNA, 11 bp, Artificial Sequence
  Comment for artificial sequence chemically pretreated Genom DNA
aaatattacat

SEQ ID NO: 77 - DNA, 11 bp, Artificial Sequence
  Comment for artificial sequence chemically pretreated Genom DNA
atttacatacc

SEQ ID NO: 78 - DNA, 11 bp, Artificial Sequence
  Comment for artificial sequence chemically pretreated Genom DNA
aatatgtaaat

SEQ ID NO: 79 - DNA, 11 bp, Artificial Sequence
  Comment for artificial sequence chemically pretreated Genom DNA
atttacatatt

SEQ ID NO: 80 - DNA, 11 bp, Artificial Sequence
  Comment for artificial sequence chemically pretreated Genom DNA
agtatgtaaat

SEQ ID NO: 81 - DNA, 11 bp, Artificial Sequence
  Comment for artificial sequence chemically pretreated Genom DNA
atttacatact

SEQ ID NO: 82 - DNA, 11 bp, Artificial Sequence
  Comment for artificial sequence chemically pretreated Genom DNA
gatatgtaaat

SEQ ID NO: 83 - DNA, 11 bp, Artificial Sequence
  Comment for artificial sequence chemically pretreated Genom DNA
atttacatatc

SEQ ID NO: 84 - DNA, 20 bp, Artificial Sequence
  Comment for artificial sequence chemically pretreated Genom DNA
ggatatgttcgggtatgttt

SEQ ID NO: 85 - DNA, 20 bp, Artificial Sequence
  Comment for artificial sequence chemically pretreated Genom DNA
aaacatacccgaacatatcc

SEQ ID NO: 86 - DNA, 20 bp, Artificial Sequence
  Comment for artificial sequence chemically pretreated Genom DNA
agatatgttcgggtatgttt

SEQ ID NO: 87 - DNA, 10 bp, Artificial Sequence
  Comment for artificial sequence chemically pretreated Genom DNA
attacgtggt

SEQ ID NO: 88 - DNA, 20 bp, Artificial Sequence
  Comment for artificial sequence chemically pretreated Genom DNA
aaacatacccgaacatatct

SEQ ID NO: 89 - DNA, 18 bp, Artificial Sequence
  Comment for artificial sequence chemically pretreated Genom DNA
tcgtttcgttttagatat

SEQ ID NO: 90 - DNA, 18 bp, Artificial Sequence
  Comment for artificial sequence chemically pretreated Genom DNA
atatctaaaacgaaacga

SEQ ID NO: 91 - DNA, 18 bp, Artificial Sequence
  Comment for artificial sequence chemically pretreated Genom DNA
atatttagagcggaacgg

SEQ ID NO: 92 - DNA, 18 bp, Artificial Sequence
  Comment for artificial sequence chemically pretreated Genom DNA
ccgttccgctctaaatat

SEQ ID NO: 93 - DNA, 10 bp, Artificial Sequence
  Comment for artificial sequence chemically pretreated Genom DNA
cgttacggtt

SEQ ID NO: 94 - DNA, 10 bp, Artificial Sequence
  Comment for artificial sequence chemically pretreated Genom DNA
aaccgtaacg

SEQ ID NO: 95 - DNA, 10 bp, Artificial Sequence
  Comment for artificial sequence chemically pretreated Genom DNA
aatcgtgacg

SEQ ID NO: 96 - DNA, 10 bp, Artificial Sequence
  Comment for artificial sequence chemically pretreated Genom DNA
cgtcacgatt

SEQ ID NO: 97 - DNA, 10 bp, Artificial Sequence
  Comment for artificial sequence chemically pretreated Genom DNA
gatcgtgacg

SEQ ID NO: 98 - DNA, 10 bp, Artificial Sequence
  Comment for artificial sequence chemically pretreated Genom DNA
accacgtaat

SEQ ID NO: 99 - DNA, 10 bp, Artificial Sequence
  Comment for artificial sequence chemically pretreated Genom DNA
cgtcacgatc

SEQ ID NO: 100 - DNA, 10 bp, Artificial Sequence
  Comment for artificial sequence chemically pretreated Genom DNA
cgttacgttt

SEQ ID NO: 101 - DNA, 10 bp, Artificial Sequence
  Comment for artificial sequence chemically pretreated Genom DNA
aaacgtaacg

SEQ ID NO: 102 - DNA, 10 bp, Artificial Sequence
  Comment for artificial sequence chemically pretreated Genom DNA
aagcgtgacg

SEQ ID NO: 103 - DNA, 10 bp, Artificial Sequence
  Comment for artificial sequence chemically pretreated Genom DNA
cgtcacgctt

SEQ ID NO: 104 - DNA, 10 bp, Artificial Sequence
  Comment for artificial sequence chemically pretreated Genom DNA
gagcgtgacg

SEQ ID NO: 105 - DNA, 10 bp, Artificial Sequence
  Comment for artificial sequence chemically pretreated Genom DNA
cgtcacgctc

SEQ ID NO: 106 - DNA, 11 bp, Artificial Sequence
  Comment for artificial sequence chemically pretreated Genom DNA
tttacgtatga

SEQ ID NO: 107 - DNA, 11 bp, Artificial Sequence
  Comment for artificial sequence chemically pretreated Genom DNA
tcatacgtaaa

SEQ ID NO: 108 - DNA, 11 bp, Artificial Sequence
  Comment for artificial sequence chemically pretreated Genom DNA
ttatgcgtgaa

SEQ ID NO: 109 - DNA, 11 bp, Artificial Sequence
  Comment for artificial sequence chemically pretreated Genom DNA
tgcgtgggcgg

SEQ ID NO: 110 - DNA, 11 bp, Artificial Sequence
  Comment for artificial sequence chemically pretreated Genom DNA
ttcacgcataa

SEQ ID NO: 111 - DNA, 11 bp, Artificial Sequence
  Comment for artificial sequence chemically pretreated Genom DNA
tttacgtttga

SEQ ID NO: 112 - DNA, 11 bp, Artificial Sequence
  Comment for artificial sequence chemically pretreated Genom DNA
tcaaacgtaaa

SEQ ID NO: 113 - DNA, 11 bp, Artificial Sequence
  Comment for artificial sequence chemically pretreated Genom DNA
ttaagcgtgaa

SEQ ID NO: 114 - DNA, 11 bp, Artificial Sequence
  Comment for artificial sequence chemically pretreated Genom DNA
ttcacgcttaa

SEQ ID NO: 115 - DNA, 11 bp, Artificial Sequence
  Comment for artificial sequence chemically pretreated Genom DNA
tttacgtttta

SEQ ID NO: 116 - DNA, 11 bp, Artificial Sequence
  Comment for artificial sequence chemically pretreated Genom DNA
taaaacgtaaa

SEQ ID NO: 117 - DNA, 11 bp, Artificial Sequence
  Comment for artificial sequence chemically pretreated Genom DNA
tgaagcgtgaa

SEQ ID NO: 118 - DNA, 11 bp, Artificial Sequence
  Comment for artificial sequence chemically pretreated Genom DNA
ttcacgcttca

SEQ ID NO: 119 - DNA, 11 bp, Artificial Sequence
  Comment for artificial sequence chemically pretreated Genom DNA
tttacgtatta

SEQ ID NO: 120 - DNA, 11 bp, Artificial Sequence
  Comment for artificial sequence chemically pretreated Genom DNA
ccgcccacgca

SEQ ID NO: 121 - DNA, 11 bp, Artificial Sequence
  Comment for artificial sequence chemically pretreated Genom DNA
taatacgtaaa

SEQ ID NO: 122 - DNA, 11 bp, Artificial Sequence
  Comment for artificial sequence chemically pretreated Genom DNA
tgatgcgtgaa

SEQ ID NO: 123 - DNA, 11 bp, Artificial Sequence
  Comment for artificial sequence chemically pretreated Genom DNA
ttcacgcatca

SEQ ID NO: 124 - DNA, 10 bp, Artificial Sequence
  Comment for artificial sequence chemically pretreated Genom DNA
aattaattaa

SEQ ID NO: 125 - DNA, 10 bp, Artificial Sequence
  Comment for artificial sequence chemically pretreated Genom DNA
ttaattaatt

SEQ ID NO: 126 - DNA, 10 bp, Artificial Sequence
  Comment for artificial sequence chemically pretreated Genom DNA
ttgattgatt

SEQ ID NO: 127 - DNA, 10 bp, Artificial Sequence
  Comment for artificial sequence chemically pretreated Genom DNA
aatcaatcaa

SEQ ID NO: 128 - DNA, 10 bp, Artificial Sequence
  Comment for artificial sequence chemically pretreated Genom DNA
tattaattaa

SEQ ID NO: 129 - DNA, 10 bp, Artificial Sequence
  Comment for artificial sequence chemically pretreated Genom DNA
ttaattaata

SEQ ID NO: 130 - DNA, 10 bp, Artificial Sequence
  Comment for artificial sequence chemically pretreated Genom DNA
ttgattgatg

SEQ ID NO: 131 - DNA, 11 bp, Artificial Sequence
  Comment for artificial sequence chemically pretreated Genom DNA
tcgtttacgta

SEQ ID NO: 132 - DNA, 10 bp, Artificial Sequence
  Comment for artificial sequence chemically pretreated Genom DNA
catcaatcaa

SEQ ID NO: 133 - DNA, 14 bp, Artificial Sequence
  Comment for artificial sequence chemically pretreated Genom DNA
ttttaaatattttt

SEQ ID NO: 134 - DNA, 14 bp, Artificial Sequence
  Comment for artificial sequence chemically pretreated Genom DNA
aaaaatatttaaaa

SEQ ID NO: 135 - DNA, 14 bp, Artificial Sequence
  Comment for artificial sequence chemically pretreated Genom DNA
gggggtgtttgggg

SEQ ID NO: 136 - DNA, 14 bp, Artificial Sequence
  Comment for artificial sequence chemically pretreated Genom DNA
ccccaaacaccccc

SEQ ID NO: 137 - DNA, 14 bp, Artificial Sequence
  Comment for artificial sequence chemically pretreated Genom DNA
ttttaaattatttt

SEQ ID NO: 138 - DNA, 14 bp, Artificial Sequence
  Comment for artificial sequence chemically pretreated Genom DNA
aaaataatttaaaa

SEQ ID NO: 139 - DNA, 14 bp, Artificial Sequence
  Comment for artificial sequence chemically pretreated Genom DNA
ggggtggtttgggg

SEQ ID NO: 140 - DNA, 14 bp, Artificial Sequence
  Comment for artificial sequence chemically pretreated Genom DNA
ccccaaaccacccc

SEQ ID NO: 141 - DNA, 14 bp, Artificial Sequence
  Comment for artificial sequence chemically pretreated Genom DNA
ttttaaattttttt

SEQ ID NO: 142 - DNA, 11 bp, Artificial Sequence
  Comment for artificial sequence chemically pretreated Genom DNA
tacgtaaacga

SEQ ID NO: 143 - DNA, 14 bp, Artificial Sequence
  Comment for artificial sequence chemically pretreated Genom DNA
aaaaaaatttaaaa

SEQ ID NO: 144 - DNA, 14 bp, Artificial Sequence
  Comment for artificial sequence chemically pretreated Genom DNA
gggggggtttgggg

SEQ ID NO: 145 - DNA, 14 bp, Artificial Sequence
  Comment for artificial sequence chemically pretreated Genom DNA
ccccaaaccccccc

SEQ ID NO: 146 - DNA, 14 bp, Artificial Sequence
  Comment for artificial sequence chemically pretreated Genom DNA
ttttaaataatttt

SEQ ID NO: 147 - DNA, 14 bp, Artificial Sequence
  Comment for artificial sequence chemically pretreated Genom DNA
aaaattatttaaaa

SEQ ID NO: 148 - DNA, 14 bp, Artificial Sequence
  Comment for artificial sequence chemically pretreated Genom DNA
ggggttgtttgggg

SEQ ID NO: 149 - DNA, 14 bp, Artificial Sequence
  Comment for artificial sequence chemically pretreated Genom DNA
ccccaaacaacccc

SEQ ID NO: 150 - DNA, 11 bp, Artificial Sequence
  Comment for artificial sequence chemically pretreated Genom DNA
gggggcggggt

SEQ ID NO: 151 - DNA, 11 bp, Artificial Sequence
  Comment for artificial sequence chemically pretreated Genom DNA
accccgccccc

SEQ ID NO: 152 - DNA, 11 bp, Artificial Sequence
  Comment for artificial sequence chemically pretreated Genom DNA
atttcgttttt

SEQ ID NO: 153 - DNA, 11 bp, Artificial Sequence
  Comment for artificial sequence chemically pretreated Genom DNA
tgcgtgggcgt

SEQ ID NO: 154 - DNA, 11 bp, Artificial Sequence
  Comment for artificial sequence chemically pretreated Genom DNA
aaaaacgaaat

SEQ ID NO: 155 - DNA, 11 bp, Artificial Sequence
  Comment for artificial sequence chemically pretreated Genom DNA
gtttcgttttt

SEQ ID NO: 156 - DNA, 11 bp, Artificial Sequence
  Comment for artificial sequence chemically pretreated Genom DNA
aaaaacgaaac

SEQ ID NO: 157 - DNA, 11 bp, Artificial Sequence
  Comment for artificial sequence chemically pretreated Genom DNA
tattattttat

SEQ ID NO: 158 - DNA, 11 bp, Artificial Sequence
  Comment for artificial sequence chemically pretreated Genom DNA
ataaaataata

SEQ ID NO: 159 - DNA, 11 bp, Artificial Sequence
  Comment for artificial sequence chemically pretreated Genom DNA
gtggggtgata

SEQ ID NO: 160 - DNA, 11 bp, Artificial Sequence
  Comment for artificial sequence chemically pretreated Genom DNA
tatcaccccac

SEQ ID NO: 161 - DNA, 11 bp, Artificial Sequence
  Comment for artificial sequence chemically pretreated Genom DNA
gattattttat

SEQ ID NO: 162 - DNA, 11 bp, Artificial Sequence
  Comment for artificial sequence chemically pretreated Genom DNA
ataaaataatc

SEQ ID NO: 163 - DNA, 11 bp, Artificial Sequence
  Comment for artificial sequence chemically pretreated Genom DNA
gtggggtgatt

SEQ ID NO: 164 - DNA, 11 bp, Artificial Sequence
  Comment for artificial sequence chemically pretreated Genom DNA
acgcccacgca

SEQ ID NO: 165 - DNA, 11 bp, Artificial Sequence
  Comment for artificial sequence chemically pretreated Genom DNA
aatcaccccac

SEQ ID NO: 166 - DNA, 10 bp, Artificial Sequence
  Comment for artificial sequence chemically pretreated Genom DNA
ttttatatgg

SEQ ID NO: 167 - DNA, 10 bp, Artificial Sequence
  Comment for artificial sequence chemically pretreated Genom DNA
ccatataaaa

SEQ ID NO: 168 - DNA, 10 bp, Artificial Sequence
  Comment for artificial sequence chemically pretreated Genom DNA
ttatataagg

SEQ ID NO: 169 - DNA, 10 bp, Artificial Sequence
  Comment for artificial sequence chemically pretreated Genom DNA
ccttatataa

SEQ ID NO: 170 - DNA, 10 bp, Artificial Sequence
  Comment for artificial sequence chemically pretreated Genom DNA
ttatatatgg

SEQ ID NO: 171 - DNA, 10 bp, Artificial Sequence
  Comment for artificial sequence chemically pretreated Genom DNA
ccatatataa

SEQ ID NO: 172 - DNA, 14 bp, Artificial Sequence
  Comment for artificial sequence chemically pretreated Genom DNA
atttttcggaaatg

SEQ ID NO: 173 - DNA, 14 bp, Artificial Sequence
  Comment for artificial sequence chemically pretreated Genom DNA
catttccgaaaaat

SEQ ID NO: 174 - DNA, 14 bp, Artificial Sequence
  Comment for artificial sequence chemically pretreated Genom DNA
tattttcgggaaat

SEQ ID NO: 175 - DNA, 11 bp, Artificial Sequence
  Comment for artificial sequence chemically pretreated Genom DNA
acgtttacgta

SEQ ID NO: 176 - DNA, 14 bp, Artificial Sequence
  Comment for artificial sequence chemically pretreated Genom DNA
atttcccgaaaata

SEQ ID NO: 177 - DNA, 14 bp, Artificial Sequence
  Comment for artificial sequence chemically pretreated Genom DNA
attttcgggaaatg

SEQ ID NO: 178 - DNA, 14 bp, Artificial Sequence
  Comment for artificial sequence chemically pretreated Genom DNA
catttcccgaaaat

SEQ ID NO: 179 - DNA, 14 bp, Artificial Sequence
  Comment for artificial sequence chemically pretreated Genom DNA
tatttttcggaaat

SEQ ID NO: 180 - DNA, 14 bp, Artificial Sequence
  Comment for artificial sequence chemically pretreated Genom DNA
atttccgaaaaata

SEQ ID NO: 181 - DNA, 14 bp, Artificial Sequence
  Comment for artificial sequence chemically pretreated Genom DNA
attttcgggaagtg

SEQ ID NO: 182 - DNA, 14 bp, Artificial Sequence
  Comment for artificial sequence chemically pretreated Genom DNA
cacttcccgaaaat

SEQ ID NO: 183 - DNA, 10 bp, Artificial Sequence
  Comment for artificial sequence chemically pretreated Genom DNA
aatagatgtt

SEQ ID NO: 184 - DNA, 10 bp, Artificial Sequence
  Comment for artificial sequence chemically pretreated Genom DNA
aacatctatt

SEQ ID NO: 185 - DNA, 10 bp, Artificial Sequence
  Comment for artificial sequence chemically pretreated Genom DNA
aatatttgtt

SEQ ID NO: 186 - DNA, 11 bp, Artificial Sequence
  Comment for artificial sequence chemically pretreated Genom DNA
tacgtaaacgt

SEQ ID NO: 187 - DNA, 10 bp, Artificial Sequence
  Comment for artificial sequence chemically pretreated Genom DNA
aacaaatatt

SEQ ID NO: 188 - DNA, 10 bp, Artificial Sequence
  Comment for artificial sequence chemically pretreated Genom DNA
aatagatggt

SEQ ID NO: 189 - DNA, 10 bp, Artificial Sequence
  Comment for artificial sequence chemically pretreated Genom DNA
accatctatt

SEQ ID NO: 190 - DNA, 10 bp, Artificial Sequence
  Comment for artificial sequence chemically pretreated Genom DNA
attatttgtt

SEQ ID NO: 191 - DNA, 10 bp, Artificial Sequence
  Comment for artificial sequence chemically pretreated Genom DNA
aacaaataat

SEQ ID NO: 192 - DNA, 13 bp, Artificial Sequence
  Comment for artificial sequence chemically pretreated Genom DNA
gggggttgacgta

SEQ ID NO: 193 - DNA, 13 bp, Artificial Sequence
  Comment for artificial sequence chemically pretreated Genom DNA
tacgtcaaccccc

SEQ ID NO: 194 - DNA, 13 bp, Artificial Sequence
  Comment for artificial sequence chemically pretreated Genom DNA
tgcgttaattttt

SEQ ID NO: 195 - DNA, 13 bp, Artificial Sequence
  Comment for artificial sequence chemically pretreated Genom DNA
aaaaattaacgca

SEQ ID NO: 196 - DNA, 13 bp, Artificial Sequence
  Comment for artificial sequence chemically pretreated Genom DNA
tacgttaattttt

SEQ ID NO: 197 - DNA, 11 bp, Artificial Sequence
  Comment for artificial sequence chemically pretreated Genom DNA
tgcgtaggcgt

SEQ ID NO: 198 - DNA, 13 bp, Artificial Sequence
  Comment for artificial sequence chemically pretreated Genom DNA
aaaaattaacgta

SEQ ID NO: 199 - DNA, 14 bp, Artificial Sequence
  Comment for artificial sequence chemically pretreated Genom DNA
tgacgtatattttt

SEQ ID NO: 200 - DNA, 14 bp, Artificial Sequence
  Comment for artificial sequence chemically pretreated Genom DNA
aaaaatatacgtca

SEQ ID NO: 201 - DNA, 14 bp, Artificial Sequence
  Comment for artificial sequence chemically pretreated Genom DNA
ggggatatgcgtta

SEQ ID NO: 202 - DNA, 14 bp, Artificial Sequence
  Comment for artificial sequence chemically pretreated Genom DNA
taacgcatatcccc

SEQ ID NO: 203 - DNA, 14 bp, Artificial Sequence
  Comment for artificial sequence chemically pretreated Genom DNA
gggggtatgcgtta

SEQ ID NO: 204 - DNA, 14 bp, Artificial Sequence
  Comment for artificial sequence chemically pretreated Genom DNA
taacgcataccccc

SEQ ID NO: 205 - DNA, 11 bp, Artificial Sequence
  Comment for artificial sequence chemically pretreated Genom DNA
cggttattttg

SEQ ID NO: 206 - DNA, 11 bp, Artificial Sequence
  Comment for artificial sequence chemically pretreated Genom DNA
caaaataaccg

SEQ ID NO: 207 - DNA, 11 bp, Artificial Sequence
  Comment for artificial sequence chemically pretreated Genom DNA
taagatggtcg

SEQ ID NO: 208 - DNA, 11 bp, Artificial Sequence
  Comment for artificial sequence chemically pretreated Genom DNA
acgcctacgca

SEQ ID NO: 209 - DNA, 11 bp, Artificial Sequence
  Comment for artificial sequence chemically pretreated Genom DNA
cgaccatctta

SEQ ID NO: 210 - DNA, 11 bp, Artificial Sequence
  Comment for artificial sequence chemically pretreated Genom DNA
tgcgtaggcgg

SEQ ID NO: 211 - DNA, 11 bp, Artificial Sequence
  Comment for artificial sequence chemically pretreated Genom DNA
ccgcctacgca

SEQ ID NO: 212 - DNA, 11 bp, Artificial Sequence
  Comment for artificial sequence chemically pretreated Genom DNA
gtaaataaata

SEQ ID NO: 213 - DNA, 11 bp, Artificial Sequence
  Comment for artificial sequence chemically pretreated Genom DNA
tatttatttac

SEQ ID NO: 214 - DNA, 11 bp, Artificial Sequence
  Comment for artificial sequence chemically pretreated Genom DNA
ataaataaaca

SEQ ID NO: 215 - DNA, 10 bp, Artificial Sequence
  Comment for artificial sequence chemically pretreated Genom DNA
aaagtaaata

SEQ ID NO: 216 - DNA, 10 bp, Artificial Sequence
  Comment for artificial sequence chemically pretreated Genom DNA
tatttacttt

SEQ ID NO: 217 - DNA, 10 bp, Artificial Sequence
  Comment for artificial sequence chemically pretreated Genom DNA
tgtttatttt

SEQ ID NO: 218 - DNA, 10 bp, Artificial Sequence
  Comment for artificial sequence chemically pretreated Genom DNA
aaaataaaca

SEQ ID NO: 219 - DNA, 10 bp, Artificial Sequence
  Comment for artificial sequence chemically pretreated Genom DNA
aatgtaaata

SEQ ID NO: 220 - DNA, 10 bp, Artificial Sequence
  Comment for artificial sequence chemically pretreated Genom DNA
tatttacatt

SEQ ID NO: 221 - DNA, 10 bp, Artificial Sequence
  Comment for artificial sequence chemically pretreated Genom DNA
tgtttatatt

SEQ ID NO: 222 - DNA, 10 bp, Artificial Sequence
  Comment for artificial sequence chemically pretreated Genom DNA
aatataaaca

SEQ ID NO: 223 - DNA, 10 bp, Artificial Sequence
  Comment for artificial sequence chemically pretreated Genom DNA
taagtaaata

SEQ ID NO: 224 - DNA, 10 bp, Artificial Sequence
  Comment for artificial sequence chemically pretreated Genom DNA
tatttactta

SEQ ID NO: 225 - DNA, 10 bp, Artificial Sequence
  Comment for artificial sequence chemically pretreated Genom DNA
tgtttattta

SEQ ID NO: 226 - DNA, 10 bp, Artificial Sequence
  Comment for artificial sequence chemically pretreated Genom DNA
taaataaaca

SEQ ID NO: 227 - DNA, 10 bp, Artificial Sequence
  Comment for artificial sequence chemically pretreated Genom DNA
tatgtaaata

SEQ ID NO: 228 - DNA, 10 bp, Artificial Sequence
  Comment for artificial sequence chemically pretreated Genom DNA
tatttacata

SEQ ID NO: 229 - DNA, 10 bp, Artificial Sequence
  Comment for artificial sequence chemically pretreated Genom DNA
tgtttatata

SEQ ID NO: 230 - DNA, 10 bp, Artificial Sequence
  Comment for artificial sequence chemically pretreated Genom DNA
tatataaaca

SEQ ID NO: 231 - DNA, 10 bp, Artificial Sequence
  Comment for artificial sequence chemically pretreated Genom DNA
taaataaata

SEQ ID NO: 232 - DNA, 10 bp, Artificial Sequence
  Comment for artificial sequence chemically pretreated Genom DNA
tatttattta

SEQ ID NO: 233 - DNA, 10 bp, Artificial Sequence
  Comment for artificial sequence chemically pretreated Genom DNA
tgtttgttta

SEQ ID NO: 234 - DNA, 10 bp, Artificial Sequence
  Comment for artificial sequence chemically pretreated Genom DNA
taaacaaaca

SEQ ID NO: 235 - DNA, 10 bp, Artificial Sequence
  Comment for artificial sequence chemically pretreated Genom DNA
tatttgttta

SEQ ID NO: 236 - DNA, 10 bp, Artificial Sequence
  Comment for artificial sequence chemically pretreated Genom DNA
taaacaaata

SEQ ID NO: 237 - DNA, 10 bp, Artificial Sequence
  Comment for artificial sequence chemically pretreated Genom DNA
gttaatgatt

SEQ ID NO: 238 - DNA, 10 bp, Artificial Sequence
  Comment for artificial sequence chemically pretreated Genom DNA
aatcattaac

SEQ ID NO: 239 - DNA, 10 bp, Artificial Sequence
  Comment for artificial sequence chemically pretreated Genom DNA
aattattaat

SEQ ID NO: 240 - DNA, 10 bp, Artificial Sequence
  Comment for artificial sequence chemically pretreated Genom DNA
attaataatt

SEQ ID NO: 241 - DNA, 10 bp, Artificial Sequence
  Comment for artificial sequence chemically pretreated Genom DNA
gttaattatt

SEQ ID NO: 242 - DNA, 10 bp, Artificial Sequence
  Comment for artificial sequence chemically pretreated Genom DNA
aataattaac

SEQ ID NO: 243 - DNA, 10 bp, Artificial Sequence
  Comment for artificial sequence chemically pretreated Genom DNA
aataattaat

SEQ ID NO: 244 - DNA, 10 bp, Artificial Sequence
  Comment for artificial sequence chemically pretreated Genom DNA
attaattatt

SEQ ID NO: 245 - DNA, 10 bp, Artificial Sequence
  Comment for artificial sequence chemically pretreated Genom DNA
gttaattaat

SEQ ID NO: 246 - DNA, 10 bp, Artificial Sequence
  Comment for artificial sequence chemically pretreated Genom DNA
attaattaac

SEQ ID NO: 247 - DNA, 10 bp, Artificial Sequence
  Comment for artificial sequence chemically pretreated Genom DNA
attaattaat

SEQ ID NO: 248 - DNA, 10 bp, Artificial Sequence
  Comment for artificial sequence chemically pretreated Genom DNA
gttaatgaat

SEQ ID NO: 249 - DNA, 10 bp, Artificial Sequence
  Comment for artificial sequence chemically pretreated Genom DNA
attcattaac

SEQ ID NO: 250 - DNA, 10 bp, Artificial Sequence
  Comment for artificial sequence chemically pretreated Genom DNA
atttattaat

SEQ ID NO: 251 - DNA, 10 bp, Artificial Sequence
  Comment for artificial sequence chemically pretreated Genom DNA
attaataaat

SEQ ID NO: 252 - DNA, 11 bp, Artificial Sequence
  Comment for artificial sequence chemically pretreated Genom DNA
aaagtgaaatt

SEQ ID NO: 253 - DNA, 11 bp, Artificial Sequence
  Comment for artificial sequence chemically pretreated Genom DNA
aatttcacttt

SEQ ID NO: 254 - DNA, 11 bp, Artificial Sequence
  Comment for artificial sequence chemically pretreated Genom DNA
ggttttatttt

SEQ ID NO: 255 - DNA, 11 bp, Artificial Sequence
  Comment for artificial sequence chemically pretreated Genom DNA
aaaataaaacc

SEQ ID NO: 256 - DNA, 11 bp, Artificial Sequence
  Comment for artificial sequence chemically pretreated Genom DNA
aaagcgaaatt

SEQ ID NO: 257 - DNA, 11 bp, Artificial Sequence
  Comment for artificial sequence chemically pretreated Genom DNA
aatttcgcttt

SEQ ID NO: 258 - DNA, 11 bp, Artificial Sequence
  Comment for artificial sequence chemically pretreated Genom DNA
ggtttcgtttt

SEQ ID NO: 259 - DNA, 11 bp, Artificial Sequence
  Comment for artificial sequence chemically pretreated Genom DNA
aaaacgaaacc

SEQ ID NO: 260 - DNA, 15 bp, Artificial Sequence
  Comment for artificial sequence chemically pretreated Genom DNA
tagttttattttttt

SEQ ID NO: 261 - DNA, 15 bp, Artificial Sequence
  Comment for artificial sequence chemically pretreated Genom DNA
aaaaaaataaaacta

SEQ ID NO: 262 - DNA, 15 bp, Artificial Sequence
  Comment for artificial sequence chemically pretreated Genom DNA
gggaaagtgaaattg

SEQ ID NO: 263 - DNA, 15 bp, Artificial Sequence
  Comment for artificial sequence chemically pretreated Genom DNA
caatttcactttccc

SEQ ID NO: 264 - DNA, 15 bp, Artificial Sequence
  Comment for artificial sequence chemically pretreated Genom DNA
ggaaaagtgaaattg

SEQ ID NO: 265 - DNA, 15 bp, Artificial Sequence
  Comment for artificial sequence chemically pretreated Genom DNA
caatttcacttttcc

SEQ ID NO: 266 - DNA, 15 bp, Artificial Sequence
  Comment for artificial sequence chemically pretreated Genom DNA
tagtttttttttttt

SEQ ID NO: 267 - DNA, 15 bp, Artificial Sequence
  Comment for artificial sequence chemically pretreated Genom DNA
aaaaaaaaaaaacta

SEQ ID NO: 268 - DNA, 15 bp, Artificial Sequence
  Comment for artificial sequence chemically pretreated Genom DNA
ggaaaagagaaattg

SEQ ID NO: 269 - DNA, 15 bp, Artificial Sequence
  Comment for artificial sequence chemically pretreated Genom DNA
caatttctcttttcc

SEQ ID NO: 270 - DNA, 15 bp, Artificial Sequence
  Comment for artificial sequence chemically pretreated Genom DNA
gggaaagagaaattg

SEQ ID NO: 271 - DNA, 15 bp, Artificial Sequence
  Comment for artificial sequence chemically pretreated Genom DNA
caatttctctttccc

SEQ ID NO: 272 - DNA, 16 bp, Artificial Sequence
  Comment for artificial sequence chemically pretreated Genom DNA
ttttaaaaataatttt

SEQ ID NO: 273 - DNA, 16 bp, Artificial Sequence
  Comment for artificial sequence chemically pretreated Genom DNA
aaaattatttttaaaa

SEQ ID NO: 274 - DNA, 16 bp, Artificial Sequence
  Comment for artificial sequence chemically pretreated Genom DNA
agggttatttttagag

SEQ ID NO: 275 - DNA, 16 bp, Artificial Sequence
  Comment for artificial sequence chemically pretreated Genom DNA
ctctaaaaataaccct

SEQ ID NO: 276 - DNA, 16 bp, Artificial Sequence
  Comment for artificial sequence chemically pretreated Genom DNA
ggagttatttttagag

SEQ ID NO: 277 - DNA, 16 bp, Artificial Sequence
  Comment for artificial sequence chemically pretreated Genom DNA
ctctaaaaataactcc

SEQ ID NO: 278 - DNA, 16 bp, Artificial Sequence
  Comment for artificial sequence chemically pretreated Genom DNA
agagttatttttagag

SEQ ID NO: 279 - DNA, 16 bp, Artificial Sequence
  Comment for artificial sequence chemically pretreated Genom DNA
ctctaaaaataactct

SEQ ID NO: 280 - DNA, 16 bp, Artificial Sequence
  Comment for artificial sequence chemically pretreated Genom DNA
ggggttatttttagag

(19) United States
(12) Patent Application Publication
(10) Pub. No.: US 2004/0248090 A1
(43) Pub. Date: Dec. 9, 2004

(54) Method for the parallel detection of the degree of methylation of genomic dna

(76) Inventors: Alexander Olek, Berlin (DE); Christian Pipenbrock, Berlin (DE)

Correspondence Address: KRIEGSMAN & KRIEGSMAN, 665 FRANKLIN STREET, FRAMINGHAM, MA, 01702, US

(21) Appl. No.: 10/149,109

(22) PCT Filed: Dec. 6, 2000
(86) PCT No.: PCT/DE00/04381

(30) Foreign Application Priority Data
Dec. 6, 1999 (DE) 199596913

(51) Int. Cl. C12Q 1/68; C12P 19/34

(52) U.S. Cl. 435/6; 435/91.2

(57) ABSTRACT

A method is described for the parallel detection of the methylation state of genomic DNA in which the following steps are conducted: a) cytosine bases unmethylated at the 5′ position in a genomic DNA sample are converted to uracil, thymidine or another base dissimilar to cytosine in its hybridization behavior; (b) of this chemically treated genomic DNA, more than ten different fragments, each of which is less than 2000 base pairs long, are amplified simultaneously by use of synthetic oligonucleotides as primers, whereby these primers each contain sequences that participate in gene regulation and/or transcribed and/or translated genomic sequences, as would be present after a treatment according to step (a); (c) the sequence context of all or a part of the CpG dinucleotides or CpNpG trinucleotides contained in the amplified fragments is determined.

The present invention concerns a method for the parallel detection of the methylation state of genomic DNA.

The levels of observation that have been well studied due to method developments in recent years in molecular biology include the genes themselves, as well as [transcription and] translation of these genes into RNA and the proteins arising therefrom. During the course of development of an individual, when a gene is turned on and how the activation and inhibition of certain genes in certain cells and tissues are controlled can be correlated with the extent and nature of the methylation of the genes or of the genome. Pathogenic states are also expressed by a modified methylation pattern of individual genes or of the genome.

The state of the art includes methods that permit the study of methylation patterns of individual genes. More recent continuing developments of these methods also permit the analysis of minimum quantities of initial material. The present invention describes a method for the parallel detection of the methylation state of genomic DNA samples, wherein a number of different fragments of sequences that participate in gene regulation or/and transcribed and/or translated sequences that are derived from one sample are amplified simultaneously and then the sequence context of CpG dinucleotides contained in the amplified fragments is investigated.

5-Methylcytosine is the most frequent covalently modified base in the DNA of eukaryotic cells. For example, it plays a role in the regulation of transcription, genomic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines is completely lost.

The modification of the genomic base cytosine to 5′-methylcytosine represents the most important and best-investigated epigenetic parameter up to the present time. Nevertheless, although there are presently methods for determining comprehensive genotypes of cells and individuals, there are no comparable approaches for generating and evaluating epigenotypic information also on a large scale.

In principle, three different basic methods are known for determining the 5-methyl status of a cytosine in the sequence context.

The first basic method is based on the use of restriction endonucleases (REs), which are “methylation-sensitive”. REs are characterized by the fact that they introduce a cleavage in the DNA at a specific DNA sequence, for the most part between 4 and 8 bases long. The position of such cleavages can then be detected by gel electrophoresis [separation], transfer onto a membrane and hybridization. [The term] methylation-sensitive means that specific bases must be present unmethylated within the recognition sequence, so that the cleavage can occur. The band pattern changes after a restriction cleavage and gel electrophoresis, depending on the methylation pattern of the DNA. Of course, the most important methylatable CpGs are found within the recognition sequences of REs, and thus cannot be investigated by this method.

The sensitivity of these methods is extremely low (Bird, A. P., and Southern, E. M., J. Mol. Biol. 118, 27-47). A variant combines PCR with these methods, and an amplification takes place by means of two primers lying on both sides of the recognition sequence after a cleavage only if the recognition sequence is present in methylated state. The sensitivity in this case theoretically increases to a single molecule of the target sequence, but, of course, single positions can be investigated only with high expenditure (Shemer, R. et al., PNAS 93, 6371-6376). It is again assumed that the methylatable position is found within the recognition sequence of a RE.

The second variant is based on partial chemical cleavage of total DNA, according to the model of a Maxam-Gilbert sequencing reaction, ligation of adaptors to the ends generated in this way, amplification with generic primers and separation by gel electrophoresis. Defined regions up to a size of less than a thousand base pairs can be investigated with this method. The method, of course, is so complicated and unreliable that it is practically no longer used (Ward, C. et al., J. Biol. Chem. 265, 3030-3033).

A relatively new method that has become the most widely used method for investigating DNA for 5-methylcytosine is based on the specific reaction of bisulfite with cytosine, which is then converted to uracil, which corresponds in its base-pairing behavior to thymidine, after subsequent alkaline hydrolysis. In contrast, 5-methylcytosine is not modified under these conditions. Thus, the original DNA is converted so that methylcytosine, which originally cannot be distinguished from cytosine by its hybridization behavior, can now be detected by “standard” molecular biology techniques as the only remaining cytosine, for example, by amplification and hybridization or sequencing. All of these techniques are based on base pairing, which can now be fully utilized. The state of the art, which concerns sensitivity, is defined by a method that incorporates the DNA to be investigated in an agarose matrix, so that the diffusion and renaturation of the DNA is prevented (bisulfite reacts only on single-stranded DNA) and all precipitation and purification steps are replaced by rapid dialysis (Olek, A. et al., Nucl. Acids Res. 24, 5064-5066). Individual cells can be investigated by this method, which illustrates the potential of the method. Of course, up until now, only individual regions of up to approximately 3000 base pairs long have been investigated; a global investigation of cells for thousands of possible methylation events is not possible. Of course, this method also cannot reliably analyze very small fragments of small sample quantities. These are lost despite the protection from diffusion through the matrix.

A review of other known methods for detecting 5-methylcytosines can also be derived from the following review article: Rein, T., DePamphilis, M. L., Zorbas, H., Nucleic Acids Res. 26, 2255 (1998).

With a few exceptions (e.g. Zeschnigk, M. et al., Eur. J. Hum. Gen. 5, 94-98; Kubota T. et al., Nat. Genet. 16, 16-17), the bisulfite technique has previously been applied only in research. However, short, specific segments of a known gene have always been amplified after a bisulfite treatment and either completely sequenced (Olek, A. and Walter, J., Nat. Genet. 17, 275-276) or individual cytosine positions are detected by a “primer extension reaction” (Gonzalgo, M. L. and Jones, P. A., Nucl. Acids Res. 25, 2529-2531) or enzyme cleavage (Xiong, Z. and Laird, P. W., Nucl. Acids Res. 25, 2532-2534). Detection by hybridization has also been described (Olek et al., WO 99/28498)

There are common features among promoters not only with respect to the presence of TATA or GC boxes, but also relative the transcription factors for which they possess binding sites and at what distance these sites are found relative to one another. The existing binding sites for a specific protein do not completely agree in their sequence, but conserved sequences of at least 4 bases are found, which can be extended by the insertion of “wobbles”, i.e., positions at which different bases are found each time. In addition, these binding sites are present at specific distances relative to one another.

The distribution of the DNA in the interphase chromatin, which occupies the greater part of the nuclear volume, however, is subject to a very special arrangement. In this case the DNA is attached at several sites to the nuclear matrix, a filamentous structure on the inside of the nuclear membrane. These regions are characterized as matrix attachment regions (MARs) or scaffold attachment regions (SARs). The attachment has a basic influence on transcription or replication. These MAR fragments do not have conservative sequences, but consist, of course, of up to 70% A or T and lie in the vicinity of cis-acting regions, which generally regulate transcription, and topoisomerase II recognition sites.

In addition to promoters and enhancers, additional regulatory elements exist for different genes, so-called insulators. These insulators can, e.g., inhibit the effect of the enhancer on the promoter, if they lie between the enhancer and the promoter, or, if they are located between heterochromatin and a gene, they protect the active gene from the influence of the heterochromatin. Examples of such insulators are: 1. so-called LCRs (locus control regions), which are comprised of several sites that are hypersensitive relative to DNAase; 2. specific sequences such as SCS (specialized chromatin structures) or SCS′, 350 or 200 bp long, respectively, and highly resistant to degradation by DNAase I and flanked on both sides by hypersensitive sites (distance of 100 bp each time). The protein BEAF-32 binds to scs′ [SCS′]. These insulators can lie on both sides of the gene.

A review of the state of the art in oligomer array production can be taken also from a special issue of Nature Genetics which appeared in January 1999, (Nature Genetics Supplement, Volume 21, January 1999), and the literature cited therein.

Patents that generally refer to the use of oligomer arrays and photolithographic mask design are, e.g., U.S. Pat. No. 5,837,832; U.S. Pat. No.5,856,174; WO-A 98/27430 and U.S. Pat. No. 5,856,101. In addition, several substance and method patents exist, which limit the use of photolabile protective groups on nucleosides, thus, e.g., WO-A 98/39348 and U.S. Pat. No. 5,763,599.

Matrix-assisted laser desorption/ionization mass spectrometery (MALDI) is a new, very powerful development for the analysis of biomolecules (Karas, M. and Hillenkamp, F. 1988. Laser desorption ionization of proteins with molecular masses exceeding 10,000 daltons. Anal. Chem. 60: 2299-2301). An analyte molecule is embedded in a matrix absorbing in the UV. The matrix is vaporized in vacuum by a short laser pulse and the analyte is thus transported unfragmented into the gas phase. An applied voltage accelerates the ions in a field-free flight tube. Ions are accelerated to variable extent based on their different masses. Smaller ions reach the detector earlier than larger ones and the flight time is converted into the mass of the ions.

Multiple fluorescently labeled probes are used for scanning an immobilized DNA array. Particularly suitable for the fluorescence label is the simple introduction of Cy3 and Cy5 dyes at the 5′OH of the respective probe. The fluorescence of the hybridized probes is detected, for example, by means of a confocal microscope. The dyes Cy3 and Cy5, in addition to many others, can be obtained commercially.

In order to calculate the expected number of amplified fragments starting from a random template DNA and two primers that are not specific for a specific positon each time, a statistical model must be established for the structure of the genome.

We indicate here the calculation of 3 models, and in this patent, of course, refer to the method described in model 3.

Model 1

In the simplest case, it is assumed that a primary DNA strand is a random sequence of four bases occurring with equal frequency. In this case, the following probability results that a perfect base pairing occurs at a given site in the genome for a random primer PrimA (of length k):

Pₐ(PrimA)=0.25^{k} (model 1 for DNA)

(this probability is the same for the sense and the anti-sense strands of the DNA).

In the case of a bisulfite treatment of the DNA, those cytosines which do not belong to a methylated CG are replaced by uracil. The base pairing behavior of uracil corresponds to that of thymine. Since CGs are very rare in DNA (less than two percent), the statistical frequency of Cs can be neglected after bisulfite treatment. The probability that for a primer PrimB (length k, of which there are a As, t Ts, g Gs and c Cs) on bisulfite-treated DNA, a perfect base pairing results, which is different for a strand treated with bisulfite and the anti-sense strand belonging thereto, and is the following:

P₁ₛ(PrimB)=0.5^{a}*0.25^{t}*0.25^{c}*0^{g} (Model 1 for bisulfite DNA strand)

P₁ₐ(PrimB)=0.25^{a}*0.5^{t}*0^{c}*0.25^{g} (Model 1 for anti-sense strand to a bisulfite DNA strand)

(If the primer contains C or G, the probability thus takes on the value 0).

Model 2:

Counts of base frequencies in DNA have shown that the four bases are not equally distributed in the DNA. Correspondingly, from DNA databases, the following frequencies (probabilities for an occurrence) of bases can be determined.

P_{DNA} (A)=0.2811

P_{DNA} (T)=0.2784

P_{DNA} (C)=0.2206

P_{DNA} (G)=0.2199

Approximately 6% of the genome of Homo sapiens from the High Throughput Sequencing Project (Database “htgs” of NIH/NCBI of Sep. 6, 1999) serves as the basis for these statistics (and the following ones for models 2 and 3). The total quantity of data amounts to more than 1.5×10⁸ base pairs, which corresponds to an estimation error of less than 10⁻⁵ for the individual probabilities.

Model 1 can be improved with the help of these values.

Thus, the probability that for a primer PrimC (length k, of which there are a As, t Ts, g Gs and c Cs) a perfect base pairing occurs is:

P₂(PrimC)=P_{DNA}(T)^{a}*P_{DNA}(A)^{t}*P_{DNA}(C)^{g}*P_{DNA}(G) (Model 3* for DNA)

sic; Model 2?—Trans. Note.

For the strand treated with bisulfite, the following probabilities result with the assumption that all CpG positions are methylated (the same statistics are obtained for the bisulfite treatment of the DNA sense and the DNA antisense strands):

P_{bDNA}(A)=0.2811

P_{bDNA}(C)=0.0140

P_{bDNA}(G)=0.2199

P_{bDNA}(T)=0.4850

The probability results that for a primer PrimD (length k, of which there are a As, t Ts, g Gs and c Cs) a perfect pairing occurs is:

P₂ₛ(PrimD)=P_{bDNA}(T)^{a}*P_{bDNA}(A)^{t}*P_{bDNA}(C)^{g}*P_{DNA}(G)^{c} (Model 3* for bisulfite DNA strand)

P₂ₐ(PrimD)=P_{bDNA}(A)^{a}*P_{bDNA}(T)^{t}*P_{bDNA}(G)^{g}*P_{DNA}(C)^{c} (Model 3* for anti-sense strand to a bisulfite DNA strand)

* sic; Model 2?—Trans. Note.

Model 3:

Basic estimating errors in model 2 result above all in the case of DNA treated with bisulfite due to the fact that C can occur only in the context CG. Model 3 considers this property and assumes that the primary DNA is a random sequence with dependence of directly adjacent bases (Markov chain of the first order). The base pairing probabilities determined emprically from the database (completely methylated; treated with bisulfite) are the same for both DNA strands, P_{bDNA} (from; to) from the following table:

| From\to | A | C | G | T |
|---|---|---|---|---|
| A | 0.0894 | 0.0033 | 0.0722 | 0.1162 |
| C | 0.0 | 0.0 | 0.0140 | 0.0 |
| G | 0.0603 | 0.0036 | 0.0601 | 0.0959 |
| T | 0.1314 | 0.0071 | 0.0736 | 0.2729 |

and for the reverse-complementary strand to this (due to corresponding exchange of inputs) P_{rbDNA} (from; to)

| From\to | A | C | G | T |
|---|---|---|---|---|
| A | 0.2729 | 0.0959 | 0.0 | 0.1162 |
| C | 0.0736 | 0.0601 | 0.0140 | 0.0722 |
| G | 0.0071 | 0.0036 | 0.0 | 0.0033 |
| T | 0.1314 | 0.0603 | 0.0 | 0.0894 |

Thus, the probability that a perfect base pairing occurs for a primer PrimE (with the base sequence B₁B₂B₃B₄ . . . ; e.g. ATTG . . . ) depends on the precise sequence of bases and results as the product:

$$\begin{matrix}{P}_{3 s} ( PrimE ) = {P}_{rbDNA} ( {B}_{1} ) \frac{{P}_{rbDNA} ( {B}_{1} ; {B}_{2} )}{{P}_{rbDNA} ( {B}_{1} )} \frac{{P}_{rbDNA} ( {B}_{2} ; {B}_{3} )}{{P}_{rbDNA} ( {B}_{2} )} \frac{{P}_{rbDNA} ( {B}_{3} ; {B}_{4} )}{{P}_{rbDNA} ( {B}_{3} )} … \\ {P}_{3 a} ( PrimE ) = {P}_{bDNA} ( {B}_{1} ) \frac{{P}_{bDNA} ( {B}_{1} ; {B}_{2} )}{{P}_{bDNA} ( {B}_{1} )} \frac{{P}_{bDNA} ( {B}_{2} ; {B}_{3} )}{{P}_{bDNA} ( {B}_{2} )} \frac{{P}_{bDNA} ( {B}_{3} ; {B}_{4} )}{{P}_{bDNA} ( {B}_{3} )} …\end{matrix}$$

(Model 3for bisulfite DNA strand)

(Model 3 for anti-sense strand to a bisulfite DNA strand)

Calculation of the Number of Amplified Fragments to be Expected:

The DNA treated with bisulfite is amplified with the use of a number of primers. From the viewpoint of the model, the DNA is comprised of a sense strand and an anti-sense strand of length of N bases (all chromosomes are summarized here). For a primer Prim, it is to be expected that the following perfect base pairings occur on the sense strand:

N*Pₛ(Prim)

The functions P₁ₛ, P₂ₛ or P₃ₛ of models 1, 2 or 3 can be utilized for this calculation, depending on the desired precision of the estimation each time. If several primers (PrimU, PrimV, PrimW, PrimX, etc.) are used simultaneously, the following results as the probability for a perfect base pairing on the sense strand at a given position:

$$\begin{matrix}{P}_{s} ( Primers ) = {P}_{s} ( PrimU ) + \\ [ ( 1 - {P}_{s} ( PrimU ) ) {P}_{s} ( PrimV ) + \\ ( 1 - {P}_{s} ( PrimU ) ) ( 1 - {P}_{s} ( PrimV ) ) {P}_{s} ( PrimW ) + \\ ( 1 - {P}_{s} ( PrimU ) ) ( 1 - {P}_{s} ( PrimV ) ) ( 1 - {P}_{s} ( PrimW ) ) {P}_{s} ( PrimX ) + \\ …\end{matrix}$$

And thus the following is the number of perfect base pairings to be expected with any of the primers:

N*Pₛ(Primers)

The analogous equations are used for the determination of Pₐ(Primers) on the anti-sense strand. An amplified product is formed precisely if a primer forms a perfect base pairing on the counterstrand within the maximum fragment length M in the case of a perfect base pairing on the sense strand. The probability of this is:

$${P}_{a} ( Primers ) {\sum }_{i = 0}^{M - 2} ( 1 - {P}_{a} ( Primers ) )$$

For large M and small Pa (Primers) this can be calculated by the following expression:

$$\frac{1 - {P}_{a} ( Primers )}{log ( 1 - {P}_{a} ( Primers ) )} [ {( 1 - {P}_{a} ( Primers ) )}^{M} - 1 ]$$

For the total number F of fragments, which are to be expected by the amplification of both strands, the following thus results:

$$\begin{matrix}F = N * {P}_{s} ( Primers ) \frac{( 1 - {P}_{a} ( Primers ) )}{log ( 1 - {P}_{a} ( Primers ) )} \\ [ {( 1 - {P}_{a} ( Primers ) )}^{M} - 1 ] + \\ N * {P}_{a} ( Primers ) \frac{( 1 - {P}_{s} ( Primers ) )}{log ( 1 - {P}_{s} ( Primers ) )} \\ [ {( 1 - {P}_{s} ( Primers ) )}^{M} - 1 ]\end{matrix}$$

This method supplies a precise expected value for predicting the number of binding sites of specific sequences to a random genomic DNA fragment that has been pretreated with bisulfite. It serves here as the basis for the calculation of the statistically expected number of amplified products in a PCR reaction starting with two primer sequences and one DNA of length N, whereby only those amplified products are considered that do not exceed a number of M nucleotides. In this patent, we proceed from the circumstance that M has the value 2000.

The known methods for the detection of cytosine methylations in genomic DNA are in principle not designed such that a multiple number of target regions in the genome to be investigated can be detected simultaneously. The object of the present invention is to create a method, with which a sample of genomic DNA can be investigated simultaneously at several positions relative to cytosine methylation.

The object is solved by the characterizing features of claim 1. Advantageous enhancements of the features are characterized in the dependent claims.

Unlike other methods, an amplification of many target regions can be produced simultaneously after chemical pretreatment of the DNA by employing appropriately adapted primer pairs. It is not absolutely necessary to know the sequence context of all of these target regions beforehand, since in many cases, as will be discussed below also by examples, consensus sequences of target regions related to the sequencing are known, which can be used for the design of specific target regions of specific or selective primer pairs, as will be described below. The method is then successfully applied, if the amplification of chemically pretreated genomic DNA supplies more fragments than can be expected statistically, each of up to a maximum of 2000 base pairs in length, of the target regions to be investigated each time.

The statistically expected value for the number of these fragments is calculated by means of the formulas described in the prior art. The number of fragments produced in the amplification step, however, can be detected by means of any molecular biological, chemical or physical methods.

For conducting the necessary statistical considerations, which are relevant also for the claims given below, the following values are assumed:

The human haploid genome contains 3 billion base pairs and 100,000 genes, which in turn encode mRNAs on average 2000 base pairs long, and the genes including the introns are on average 15,000 base pairs long. Promoters comprise on average 1000 base pairs per gene. Thus if the statistically expected value for the number of amplified products, which lie in transcribed sequences starting from two primers, is to be calculated, then first the expected value for the total genome is to be calculated according to the above formula (method 3) and then is to be calculated with the fraction of transcribed sequences on the total genome. We proceed analogously for parts of any genome as well as for promoters and translated sequences (coding mRNA).

The present invention thus describes a method for the parallel detection of the methylation state of genomic DNA. Thus, several cytosine methylations will be analyzed simultaneously in a DNA sample. For this purpose, the following method steps are sequentially conducted:

First, a genomic DNA sample is chemically treated in such a way that cytosine bases unmethylated at the 5′ position are converted to uracil, thymine or another base dissimilar to cytosine in its hybridizing behavior. Preferably, the above-described treatment of genomic DNA with bisultite (hydrogen sulfite, disulfite) and subsequent alkaline hydrolysis will be used for this purpose, which leads to the conversion of unmethylated cytosine nucleobases to uracil.

In a second step of the method, more than ten different fragments of the pretreated genomic DNA are amplified simultaneously by use of synthetic oligonucleotides as primers, whereby more than twice as many fragments as statistically to be expected originate from transcribed and/or translated sequences or sequencers that participate in gene regulation. This can be achieved by means of different methods.

In a preferred variant of the method, at least one of the oligonucleotides used for the ampification contains fewer nucleobases than would be necessary statistically for a sequence-specific hybridization to the chemically treated genomic DNA sample, which can lead to the amplification of several fragments simultaneously. In this case, the total number of nucleobases contained in this oligonucleotide is less than 17. In a particularly preferred variant of the method, the number of nucleobases contained in this oligonucleotide is less than 14.

In another preferred variant of the method, more than 4 oligonucleotides with different sequence are used simultaneously for the amplification in one reaction vessel. In a particularly preferred variant, more than 26 different oligonucleotides are used simultaneously for the production of a complex amplified product. In a particularly preferred variant of the method, more than double the number of fragments that is statistically to be expected originate from genomic segments that participate in the regulation of genes, e.g., promoters and enhancers, than would be expected in a purely random selection of oligonucleotides sequences. In another particularly preferred variant of the method, more than double the number of amplified fragments originate from genomic segments that are transcribed into mRNA in at least one cell of the respective organism, or from placed genomic segments after transcription into mRNA (exons), than would be expected in the case of a purely random selection of oligonucleotide sequences.

In another particularly preferred variant of the method, more than double the number of amplified fragments originate from genomic segments that code for parts of one or more gene families, or they originate from genomic segments that contain sequences characteristic of so-called “matrix attachment sites” (MARS) than would be expected in a purely random selection of oligonucleotide sequences.

In another particularly preferred variant of the method, more than double the number of amplified segments originate from genomic segments that organize the packing density of the chromatin as so-called “boundary elements” or they originate from multiple drug resistant gene (MDR) promoters or coding regions, than would be expected in the case of a purely random selection of oligonucleotide sequences.

In another particularly preferred variant of the method, two oligonucleotides or two classes of oligonucleotides are used for the amplification of the described fragments, one of which or one class of which can contain the base C, but not the base G, the context CpG or CpNpG, and the other of which or the other class of which may contain the base G, but not the base C, except in the context CpG or CpNpG.

In another preferred variant of the method, the amplification is conducted by means of two oligonucleotides, one of which contains a sequence four to sixteen bases long, which is complementary or corresponds to a DNA that would be formed if a DNA fragment of the same length, to which one of the following factors binds:

| | |
|---|---|
| AhR/Arnt | aryl hydrocarbon receptor/aryl hydro- |
| | carbon receptor nuclear translocator |
| Arnt | aryl hydrocarbon receptor nuclear |
| | translocator |
| AML-1a | CBFA2; core-binding factor, runt domain, |
| | alpha subunit 2 (acute myeloid leukemia |
| | 1; aml1 oncogene) |
| AP-1 | activator protein-1 (AP-1); Synonyme: |
| | c-Jun |
| C/EBP | CCAAT/enhancer binding protein |
| C/EBPalpha | CCAAT/enhancer binding protein (C/EBP), |
| | alpha |
| C/EBPbeta | CCAAT/enhancer binding protein (C/EBP), |
| | beta |
| CDP | CUTL1; cut (Drosophila)-like 1 (CCAAT |
| | displacement protein) |
| CDP | CUTL1; cut (Drosophila)-like 1 (CCAAT |
| | displacement protein) |
| CDP CR1 | complement component (3b/4b) receptor 1 |
| CDP CR3 | complement component (3b/4b) receptor 3 |
| CHOP-C/ | DDIT; DNA-damage-inducible transcript |
| EBPalpha | 3/CCAAT/enhancer binding protein |
| | (C/EBP), alpha |
| c-Myc/Max | avian myelocytomatosis viral oncogene/ |
| | MYC-ASSOCIATED FACTOR X |
| CREB | cAMP responsive element binding protein |
| CRE-BP1 | CYCLIC AMP RESPONSE ELEMENT-BINDING |
| | PROTEIN 2, CREB2, CREBP1; now ATF2; |
| | activating transcription factor 2 |
| CRE-BP1/ | activator protein-1 (AP-1); Synonyme: |
| c-Jun | c-Jun |
| CREB | MP responsive element binding protein |
| E2F | E2F transcription factor (originally |
| | identified as a DNA-binding protein |
| | essential E1A-dependent activation of |
| | the adenovirus E2 promoter) |
| E47 | transcription factor 3 (E2A immuno- |
| | globulin enhancer binding factors |
| | E12/E47) |
| E47 | transcription factor 3 (E2A immuno- |
| | globulin enhancer binding factors |
| | E12/E47) |
| Egr-1 | early growth response 1 |
| Egr-2 | early growth response 2 (Krox-20 |
| | (Drosophila) homolog) |
| ELK-1 | ELK1, member of ETS (environmental |
| | tobacco smoke) oncogene family |
| Freac-2 | FKHL6; forkhead (Drosophila)-like 6; |
| | FORKHEAD-RELATED ACTIVATOR 2; FREAC2 |
| Freac-3 | FKHL7; forkhead (Drosophila)-like 7; |
| | FORKHEAD-RELATED ACTIVATOR 3; FREAC3 |
| Freac-4 | FKHL8; forkhead (Drosophila)-like 8; |
| | FORKHEAD-RELATED ACTIVATOR 4; FREAC4 |
| Freac-7 | FKHL11; forkhead (Drosophila)-like 9; |
| | FORKHEAD-RELATED ACTIVATOR 7; FREAC7 |
| GATA-1 | GATA-binding protein 1/Enhancer-Binding |
| | Protein GATA1 |
| GATA-1 | GATA-binding protein 1/Enhancer-Binding |
| | Protein GATA1 |
| GATA-1 | GATA-binding protein 1/Enhancer-Binding |
| | Protein GATA1 |
| GATA-2 | GATA-binding protein 2/Enhancer-Binding |
| | Protein GATA2 |
| GATA-3 | GATA-binding protein 3/Enhancer-Binding |
| | Protein GATA3 |
| GATA-X | |
| HFH-3 | FKHL10; forkhead (Drosophila)-like 10; |
| | FORKHEAD-RELATED ACTIVATOR 6; FREAC6 |
| HNF-1 | TCF1; transcription factor 1, hepatic; |
| | LF-B1, hepatic nuclear factor (HNF1), |
| | albumin proximal factor |
| HNF-4 | hepatocyte nuclear factor 4 |
| IRF-1 | interferon regulatory factor 1 |
| ISRE | interferon-stimulated response element |
| Lmo2 | LIM domain only 2 (rhombotin-like 1) |
| complex | |
| MEF-2 | MADS box transcription enhancer factor |
| | 2, polypeptide A (myocyte enhancer |
| | factor 2A) |
| MEF-2 | MADS box transcription enhancer factor |
| | 2, polypeptide A (myocyte enhancer |
| | factor 2A) |
| myogenin/ | Myogenin (myogenic factor 4)/Neuro- |
| NF-1 | fibromin 1; NEUROFIBROMATOSIS, TYPE I |
| MZF1 | ZNF42; zinc finger protein 42 |
| | (myeloid-specific retinoic acid- |
| | responsive) |
| MZF1 | ZNF42; zinc finger protein 42 |
| | (myeloid-specific retinoic acid- |
| | responsive) |
| NF-E2 | NFE2; nuclear factor (erythroid- |
| | derived 2), 45 kD |
| NF-kappaB | nuclear factor of kappa light poly- |
| (p50) | peptide gene enhancer in B-cells p50 |
| | subunit |
| NF-kappaB | nuclear factor of kappa light poly- |
| (p65) | peptide gene enhancer in B-cells p65 |
| | subunit |
| NF-kappaB | nuclear factor of kappa light poly- |
| | peptide gene enhancer in B-cells |
| NF-kappaB | nuclear factor of kappa light poly- |
| | peptide gene enhancer in B-cells |
| NRSF | NEURON RESTRICTIVE SILENCER FACTOR; |
| | REST; RE1-silencing transcription |
| | factor |
| Oct-1 | OCTAMER-BINDING TRANSCRIPTION FACTOR 1; |
| | POU2F1; POU domain, class 2, |
| | transcription factor 1 |
| Oct-1 | OCTAMER-BINDING TRANSCRIPTION FACTOR 1; |
| | POU2F1; POU domain, class 2, |
| | transcription factor 1 |
| Oct-1 | OCTAMER-BINDING TRANSCRIPTION FACTOR 1; |
| | POU2F1; POU domain, class 2, |
| | transcription factor 1 |
| Oct-1 | OCTAMER-BINDING TRANSCRIPTION FACTOR 1; |
| | POU2F1; POU domain, class 2, |
| | transcription factor 1 |
| Oct-1 | OCTAMER-BINDING TRANSCRIPTION FACTOR 1; |
| | POU2F1; POU domain, class 2, |
| | transcription factor 1 |
| P300 | E1A (adenovirus E1A oncoprotein)- |
| | BINDING PROTEIN, 300-KD |
| P53 | tumor protein p53 (Li-Fraumeni |
| | syndrome); TP53 |
| Pax-1 | paired box gene 1 |
| Pax-3 | paired box gene 3 (Waardenburg |
| | syndrome 1) |
| Pax-6 | paired box gene 6 (aniridia, keratitis) |
| Pbx 1b | pre-B-cell leukemia transcription factor |
| Pbx-1 | pre-B-cell leukemia transcription factor 1 |
| RORalpha2 | RAR-RELATED ORPHAN RECEPTOR ALPHA; |
| | RETINOIC ACID-BINDING RECEPTOR ALPHA |
| RREB-1 | ras responsive element binding protein 1 |
| SP1 | simian-virus-40-protein-1 |
| SP1 | simian-virus-40-protein-1 |
| SREBP-1 | sterol regulatory element binding |
| | transcription factor 1 |
| SRF | serum response factor (c-fos serum |
| | response element-binding transcription |
| | factor) |
| SRY | sex determining region Y |
| STAT3 | signal transducer and activator of |
| | transcription 1, 91 kD |
| Tal-1al- | T-cell acute lymphocytic leukemia |
| pha/E47 | 1/transcription factor 3 (E2A immuno- |
| | globulin enhancer binding factors |
| | E12/E47) |
| TATA | cellular and viral TATA box elements |
| Tax/CREB | Transiently-expressed axonal glyco- |
| | protein/cAMP responsive element binding |
| | protein |
| Tax/CREB | Transiently-expressed axonal glyco- |
| | protein/cAMP responsive element binding |
| | protein |
| TCF11/MafG | v-maf musculoaponeurotic fibrosarcoma |
| | (avian) oncogene family, protein G |
| TCF11 | Transcription Factor 11; TCF11; NFE2L1; |
| | nuclear factor (erythroid-derived |
| | 2)-like 1 |
| USF | upstream stimulating factor |
| Whn | winged-helix nude |
| X-BP-1 | X-box binding protein 1 oder |
| YY1 | ubiquitously distributed transcription |
| | factor belonging to theGLI-Kruppel |
| | class of zinc finger proteins |

would be chemically treated such that cytosine bases unmethylated in the 5′-position are converted to uracil, thymidine or another base dissimiliar to cytosine in its hybridization behaviour.

In another preferred variant of the method, the amplification is conducted by means of two oligonucleotides or two classes of oligonucleotides, one of which or one class of which contains the sequence that is four to sixteen bases long, which is complementary or corresponds to a DNA that would be formed if a DNA fragment of the same length, which can bring about the specific localization of genome/chromatin segments within the cell nucleus by means of its sequence or secondary structure, would be chemically treated such that cytosine bases that are unmethylated at the 5′ position will be converted to uracil, thymidine or another base dissimilar to cytosine in its hybridization behaviour.

In another preferred variant of the method, the amplification is conducted by means of two oligonucleotides or two classes of oligonucleotides, one of which or one class of which contains one of the sequences:

TCGCGTGTA, TACACGCGA, TGTACGCGA, TCGCGTACA,
TTGCGTGTT, AACACGCAA, GGTACGTAA, TTACGTACC,
TCGCGTGTT, AACACGCGA, GGTACGCGA, TCGCGTACC,
TTGCGTGTA, TACACGCAA, TGTACGTAA, TTACGTACA,
TACGTG, CACGTA, TACGTG, CACGTA,
ATTGCGTGT, ACACGCAAT, GTACGTAAT, ATTACGTAC,
ATTGCGTGA, TCACGCAAT, TTACGTAAT, ATTACGTAA,
ATCGCGTGA, TCACGCGAT, TTACGCGAT, ATCGCGTAA,
ATCGCGTGT, ACACGCGAT, GTACGCGAT, ATCGCGTAC,
TGTGGT, ACCACA, ATTATA, TATAAT,
TGAGTTAG, CTAACTCA, TTGATTTA, TAAATCAA,
TGATTTAG, CTAAATCA, TTGAGTTA, TAACTCAA,
TTTGGT, ACCAAA, ATTAAA, TTTAAT,
TGTGGA, TCCACA, TTTATA, TATAAA,
TTTGGA, TCCAAA, TTTAAA, TTTAAA,
TGTGGT, ACCACA, ATTATA, TATAAT,
ATTAT, ATAAT, GTAAT, ATTAC,
ATTGT, ACAAT, GTAAT, ATTAC,
GAAAG, CTTTC, TTTTT, AAAAA,
GTAAT, ATTAC, ATTGT, ACAAT,
GAAAT, ATTTC, ATTTT, AAAAT,
GTAAG, CTTAC, TTTGT, ACAAA,
TTAATAATCGAT, ATCGATTATTAA, ATCGATTATTGG,
CCAATAATCGAT, ATCGATTA, TAATCGAT, TAATCGAT,
ATCGATTA,
ATCGATCGG, CCGATCGAT, TCGATCGAT, ATCGATCGA,
ATCGATCGT, ACGATCGAT, GCGATCGAT, ATCGATCGC,
TATCGATA, TATCGATA, TATCGGTG, CACCGATA,
TATTAATA, TATTAATA, TATTGGTG, CACCAATA,
GTGTAATATTT, AAATATTACAC, GGGTATTGTAT,
ATACAATACCC, GTGTAATTTTT, AAAAATTACAC,
GGGGATTGTAT, ATACAATCCCC, ATGTAATTTTT,
AAAAATTACAT, GGGGATTGTAT, ATACAATCCCC,
ATGTAATATTT, AAATATTACAT, GGGTATTGTAT,
ATACAATACCC, ATTACGTGGT, ACCACGTAAT, ATTACGTGGT,
ACCACGTAAT, TGACGTAA, TTACGTCA, TTACGTTA,
TAACGTAA, TGACGTTA, TAACGTCA, TGACGTTA, TAACGTCA,
TTACGTAA, TTACGTAA, TTACGTAA, TTACGTAA, TGACGTTA,
TAACGTCA, TAACGTTA, TAACGTTA,
TGACGT, ACGTCA, GCGTTA, TAACGC,
TGACGT, ACGTCA, ACGTTA, TAACGT,
TTTCGCGT, ACGCGAAA, GCGCGAAA, TTTCGCGC,
TTTGGCGT, ACGCCAAA, GCGTTAAA, TTTAACGC,
TAGGTGTTA, TAACACCTA, TAATATTTG, CAAATATTA,
TAGGTGTTT, AAACACCTA, GAATATTTG, CAAATATTC,
GTAGGTGG, CCACCTAC, TTATTTGT, ACAAATAA,
GTAGGTGT, ACACCTAC, ATATTTGT, ACAAATAT,
TGCGTGGGCGG, CCGCCCACGCA, TCGTTTACGTA,
TACGTAAACGA, TGCGTGGGCGT, ACGCCCACGCA,
ACGTTTACGTA, TACGTAAACGT,
TGCGTAGGCGT, ACGCCTACGCA, ACGTTTACGTA,
TACGTAAACGT, TGCGTAGGCGG, CCGCCTACGCA,
TCGTTTACGTA, TACGTAAACGA, ATAGGAAGT, ACTTCCTAT,
ATTTTTTGT, ACAAAAAAT,
TCGGAAGT, ACTTCCGA, ATTTTCGG, CCGAAAAT,
TCGGAAGT, ACTTCCGA, GTTTTCGG, CCGAAAAC,
TCGGAAAT, ATTTCCGA, ATTTTCGG, CCGAAAAT,
TCGGAAAT, ATTTCCGA, GTTTTCGG, CCGAAAAC,
GTAAATAA, TTATTTAC, TTGTTTAT, ATAAACAA,
GTAAATAAATA, TATTTATTTAC, TGTTTATTTAT,
ATAAATAAACA,
AAAGTAAATA, TATTTACTTT, TGTTTATTTT, AAAATAAACA,
AATGTAAATA, TATTTACATT, TGTTTATATT, AATATAAACA,
TAAGTAAATA, TATTTACTTA, TGTTTATTTA, TAAATAAACA,
TATGTAAATA, TATTTACATA, TGTTTATATA, TATATAAACA,
ATAAATA, TATTTAT, TGTTTAT, ATAAACA,
ATAAATA, TATTTAT, TATTTAT, ATAAATA,
GATA, TATC, TATT, AATA,
TAGATAA, TTATCTA, TTATTTG, CAAATAA,
TTGATAA, TTATCAA, TTATTAG, CTAATAA,
GATAA, TTATC, TTATT, AATAA,
GATG, CATC, TATT, AATA,
GATAG, CTATC, TTATT, AATAA,
GATAAG, CTTATC, TTTATT, AATAAA,
TGTTTATTTA, TAAATAAACA, TAAATAAATA, TATTTATTTA,
TGTTTGTTTA, TAAACAAACA, TAAATAAATA, TATTTATTTA,
TATTTATTTA, TAAATAAATA, TAAATAAATA, TATTTATTTA,
TATTTGTTTA, TAAACAAATA, TAAATAAATA, TATTTATTTA,
GTTAATGATT, AATCATTAAC, AATTATTAAT, ATTAATAATT,
GTTAATTATT, AATAATTAAC, AATAATTAAT, ATTAATTATT,
GTTAATTAAT, ATTAATTAAC, ATTAATTAAT, ATTAATAAAT,
GTTAATGAAT, ATTCATTAAC, ATTTATTAAT, ATTAATAAAT,
TAAAGTTTA, TAAACTTTA, TGAATTTTG, CAAAATTCA,
TAAAGGTTA, TAACCTTTA, TGATTTTTG, CAAAAATCA,
AAAGTGAAATT, AATTTCACTTT, GGTTTTATTTT,
AAAATAAAACC, AAAGCGAAATT, AATTTCGCTTT,
GGTTTCGTTTT, AAAACGAAACC,
TAGTTTTATTTTTTT, AAAAAAATAAAACTA, GGGAAAGTGAAATTG,
CAATTTCACTTTCCC,
TAGTTTTATTTTTTT, AAAAAAATAAAACTA, GGAAAAGTGAAATTG,
CAATTTCACTTTTCC,
TAGTTTTTTTTTTTT, AAAAAAAAAAAACTA, GGAAAAGAGAAATTG,
CAATTTCTCTTTTCC,
TAGTTTTTTTTTTTT, AAAAAAAAAAAACTA, GGGAAAGAGAAATTG,
CAATTTCTCTTTCCC,
TAGGTG, CACCTA, TATTTG, CAAATA,
TTTTAAAAATAATTTT, AAAATTATTTTTAAAA,
AGGGTTATTTTTAGAG, CTCTAAAAATAACCCT,
TTTTAAAAATAATTTT, AAAATTATTTTTAAAA,
GGAGTTATTTTTAGAG, CTCTAAAAATAACTCC,
TTTTAAAAATAATTTT, AAAATTATTTTTAAAA,
AGAGTTATTTTTAGAG, CTCTAAAAATAACTCT,
TTTTAAAAATAATTTT, AAAATTATTTTTAAAA,
GGGGTTATTTTTAGAG, CTCTAAAAATAACCCC,
TGTTATTAAAAATAGAAA, TTTCTATTTTTAATAACA,
TTTTTATTTTTAGTAATA, TATTACTAAAAATAAAAA,
TGTTATTAAAAATAGAAT, ATTCTATTTTTAATAACA,
GTTTTATTTTTAGTAATA, TATTACTAAAAATAAAAC,
TTTGGTAT, ATACCAAA, GTGTTAAA, TTTAACAC
GGGGA, TCCCC, TTTTT, AAAAA,
TAGGGG, CCCCTA, TTTTTA, TAAAAA,
GAGGGG, CCCCTC, TTTTTT, AAAAAA,
TGTTGAGTTAT, ATAACTCAACA, ATGATTTAGTA,
TACTAAATCAT, TGTTGATTTAT, ATAAATCAACA,
GTGAGTTAGTA, TACTAACTCAC, TGTTGAGTTAT,
ATAACTCAACA, ATGATTTAGTA, TACTAAATCAT,
TGTTGATTTAT, ATAAATCAACA, GTGAGTTAGTA,
TACTAACTCAC, GGGGATTTTT, AAAAATCCCC, GGGAATTTTT,
AAAAATTCCC, GGGGATTTTT, AAAAATCCCC, GGGGATTTTT,
AAAAATCCCC, GGGGATTTTT, AAAAATCCCC, GGAAATTTTT,
AAAAATTTCC, GGGAATTTTT, AAAAATTCCC, GGAAATTTTT,
AAAAATTTCC, GGGAATTTTT, AAAAATTCCC, GGAAATTTTT,
AAAAATTTCC, GGGATTTTTT, AAAAAATCCC, GGAAAGTTTT,
AAAACTTTCC, GGGAATTTTT, AAAAATTCCC, GGGAATTTTT,
AAAAATTCCC, GGGATTTTTT, AAAAAATCCC, GGGAAGTTTT,
AAAACTTCCC, GGGATTTTTTA, TAAAAAATCCC, TGGAAAGTTTT,
AAAACTTTCCA, TTTAGTATTACGGATAGAGGT,
ACCTCTATCCGTAATACTAAA, GTTTTTGTTCGTGGTGTTGAA,
TTCAACACCACGAACAAAAAC, TTTAGTATTACGGATAGAGTT,
AACTCTATCCGTAATACTAAA, GGTTTTGTTCGTGGTGTTGAA,
TTCAACACCACGAACAAAACC, TTTAGTATTACGGATAGCGTT,
AACGCTATCCGTAATACTAAA, GGCGTTGTTCGTGGTGTTGAA,
TTCAACACCACGAACAACGCC, TTTAGTATTACGGATAGCGGT,
ACCGCTATCCGTAATACTAAA, GTCGTTGTTCGTGGTGTTGAA,
TTCAACACCACGAACAACGAC,
ATATGTAAAT, ATTTACATAT, ATTTGTATAT, ATATACAAAT,
TTATGTAAAT, ATTTACATAA, ATTTGTATAA, TTATACAAAT,
GAATATTTA, TAAATATTC, TGAATATTT, AAATATTCA,
GAATATGTA, TACATATTC, TGTATATTT, AAATATACA,
ATAAT, ATTAT, ATTAT, ATAAT,
GTAAT, ATTAC, ATTAT, ATAAT,
AATGTAAAT, ATTTACATT, ATTTGTATT, AATACAAAT,
ATTTGTATATT, AATATACAAAT, GGTATGTAAAT,
ATTTACATACC, ATTTGTATATT, AATATACAAAT,
AATATGTAAAT, ATTTACATATT, ATTTGTATATT,
AATATACAAAT, AGTATGTAAAT, ATTTACATACT,
ATTTGTATATT, AATATACAAAT, GATATGTAAAT,
ATTTACATATC,
AGGAGT, ACTCCT, ATTTTT, AAAAAT,
GGGAGT, ACTCCC, ATTTTT, AAAAAT,
GGATATGTTCGGGTATGTTT, AAACATACCCGAACATATCC,
GGATATGTTCGGGTATGTTT, AAACATACCCGAACATATCC,
GGATATGTTCGGGTATGTTT, AAACATACCCGAACATATCC,
AGATATGTTCGGGTATGTTT, AAACATACCCGAACATATCT,
TCGTTTCGTTTTAGATAT, ATATCTAAAACGAAACGA,
ATATTTAGAGCGGAACGG, CCGTTCCGCTCTAAATAT,
CGTTACGGTT, AACCGTAACG, AATCGTGACG, CGTCACGATT,
CGTTACGGTT, AACCGTAACG, GATCGTGACG, CGTCACGATC,
CGTTACGTTT, AAACGTAACG, AAGCGTGACG, CGTCACGCTT,
CGTTACGTTT, AAACGTAACG, GAGCGTGACG, CGTCACGCTC,
TTTACGTATGA, TCATACGTAAA, TTATGCGTGAA,
TTCACGCATAA, TTTACGTTTGA, TCAAACGTAAA,
TTAAGCGTGAA, TTCACGCTTAA, TTTACGTTTTA,
TAAAACGTAAA, TGAAGCGTGAA, TTCACGCTTCA,
TTTACGTATTA, TAATACGTAAA, TGATGCGTGAA,
TTCACGCATCA,
AATTAATTAA, TTAATTAATT, TTGATTGATT, AATCAATCAA,
TATTAATTAA, TTAATTAATA, TTGATTGATG, CATCAATCAA,
TAATTAT, ATAATTA, ATGATTG, CAATCAT,
TAGGTTA, TAACCTA, TGATTTA, TAAATCA,
TTTTAAATATTTTT, AAAAATATTTAAAA, GGGGGTGTTTGGGG,
CCCCAAACACCCCC,
TTTTAAATTATTTT, AAAATAATTTAAAA, GGGGTGGTTTGGGG,
CCCCAAACCACCCC,
TTTTAAATTTTTTT, AAAAAAATTTAAAA, GGGGGGGTTTGGGG,
CCCCAAACCCCCCC,
TTTTAAATAATTTT, AAAATTATTTAAAA, GGGGTTGTTTGGGG,
CCCCAAACAACCCC,
GAGGCGGGG, CCCCGCCTC, TTTCGTTTT, AAAACGAAA,
GAGGTAGGG, CCCTACCTC, TTTTGTTTT, AAAACAAAA,
AAGGCGGGG, CCCCGCCTT, TTTCGTTTT, AAAACGAAA,
AAGGTAGGG, CCCTACCTT, TTTTGTTTT, AAAACAAAA,
GGGGGCGGGGT, ACCCCGCCCCC, ATTTCGTTTTT,
AAAAACGAAAT, GGGGGCGGGGT, ACCCCGCCCCC,
GTTTCGTTTTT, AAAAACGAAAC, TATTATTTTAT,
ATAAAATAATA, GTGGGGTGATA, TATCACCCCAC,
GATTATTTTAT, ATAAAATAATC, GTGGGGTGATT,
AATCACCCCAC,
ATTACGTGAT, ATCACGTAAT, ATTACGTGAT, ATCACGTAAT,
ATTACGTGAT, ATCACGTAAT, GTTACGTGAT, ATCACGTAAC,
TTTTATATGG, CCATATAAAA, TTATATAAGG, CCTTATATAA,
TTATATATGG, CCATATATAA, TTATATATGG, CCATATATAA,
AAATAAT, ATTATTT, GTTGTTT, AAACAAC,
AAATTAA, TTAATTT, TTAGTTT, AAACTAA,
AAATTAT, ATAATTT, GTAGTTT, AAACTAC,
AAATAAA, TTTATTT, TTTGTTT, AAACAAA,
ATTTTTCGGAAATG, CATTTCCGAAAAAT, TATTTTCGGGAAAT,
ATTTCCCGAAAATA,
ATTTTTCGGAAATG, CATTTCCGAAAAAT, TATTTTCGGGAAAT,
ATTTCCCGAAAATA,
ATTTTCGGGAAATG, CATTTCCCGAAAAT, TATTTTTCGGAAAT,
ATTTCCGAAAAATA,
ATTTTCGGGAAGTG, CACTTCCCGAAAAT, TATTTTTCGGAAAT,
ATTTTCCGAAAAATA,
AATAGATGTT, AACATCTATT, AATATTTGTT, AACAAATATT,
AATAGATGGT, ACCATCTATT, ATTATTTGTT, AACAAATAAT,
GTATAAATA, TATTTATAC, TATTTATAT, ATATAAATA,
GTATAAATG, CATTTATAC, TATTTATAT, ATATAAATA,
GTATAAAAA, TTTTTATAC, TTTTTATAT, ATATAAAAA,
GTATAAAAG, CTTTTATAC, TTTTTATAT, ATATAAAAA,
TTATAAATA, TATTTATAA, TATTTATAG, CTATAAATA,
TTATAAATG, CATTTATAA, TATTTATAG, CTATAAATA,
TTATAAAAA, TTTTTATAA, TTTTTATAG, CTATAAAAA,
TTATAAAAG, CTTTTATAA, TTTTTATAG, CTATAAAAA,
GGGGGTTGACGTA, TACGTCAACCCCC, TGCGTTAATTTTT,
AAAAATTAACGCA,
GGGGGTTGACGTA, TACGTCAACCCCC, TACGTTAATTTTT,
AAAAATTAACGTA,
TGACGTATATTTTT, AAAAATATACGTCA, GGGGATATGCGTTA,
TAACGCATATCCCC,
TGACGTATATTTTT, AAAAATATACGTCA, GGGGGTATGCGTTA,
TAACGCATACCCCC,
ATGATTTAGTA, TACTAAATCAT, TGTTGAGTTAT,
ATAACTCAACA, GTTAT, ATAAC, ATGAT, ATCAT,
TTACGTGA, TCACGTAA, TTACGTGG, CCACGTAA,
TTACGTGG, CCACGTAA, TTACGTGG, CCACGTAA,
TTACGTGG, CCACGTAA, TTACGTGA, TCACGTAA,
TTACGTGA, TCACGTAA, TTACGTGA, TCACGTAA,
GACGTT, AACGTC, AGCGTT, AACGCT,
TGACGTGT, ACACGTCA, ATACGTTA, TAACGTAT,
TGACGTGG, CCACGTCA, TTACGTTA, TAACGTAA,
CGGTTATTTTG, CAAAATAACCG, TAAGATGGTCG oder
CGACCATCTTA

which is complementary or corresponds to a DNA that would be formed if a DNA fragment of the same length, which can bring about the specific localization of genome/chromatin segments within the cell nucleus by means of its sequence or secondary structure, would be chemically treated in such a way that cytosine bases unmethylated at the 5′ position would be converted into uracil, thymidine or another base dissimiliar to cytosine in its hybridization behavior.

In a particularly preferred variant of the method, the oligonucleotides used for the amplification contain several positions, except in the above-defined consensus sequences, at which either any of the three bases G, A and T or any of the three bases C, A and T can be present.

In a particularly preferred variant of the method, the oligonucleotides used for the amplification contain, except in one of the above-described consensus sequences, only a maximum addition of as many other bases as is necessary for the simultaneous amplification of more than one hundred different fragments for each reaction of the DNA chemically treated as above.

In a third step of the method, the sequence context of all or one part of the CpG dinucleotides or CpNpG trinucleotides contained in the amplified fragments is investigated.

In a particularly preferred variant of the method, analysis is conducted by hybridizing the fragments already provided with a fluorescence marker in the amplification to an oligonucleotide array (DNA chip). The fluorescence marker may be introduced either by means of the primers used or by a fluorescently labeled nucleotide (e.g., Cy5-dCTP, which can be obtained commercially from Amersham-Pharmacia).

Complementary fragments hybridize to the respective oligomers immobilized on the chip surface, and non-complementary fragments are removed in one or more washing steps. The fluorescence at the respective sites of hybridization on the chip then permits a conclusion on the sequence context of the CpG dinucleotides or CpNpG trinucleotides contained in the amplfied fragments.

In another preferred variant of the method, the amplified fragments are immobilized on a surface and then a hybridization is conducted with a combinatory library of distinguishable oligonucleotide or PNA oligomer probes. Again, uncomplementary probes are removed by one or more washing steps. The hybridized probes are detected either by means of their fluorescent markers or, in a particularly preferred variant of the method, they are detected by means of matrix-assisted laser desorption/ionization mass spectrometry (MALDI-MS) on the basis of their unequivocal mass. Probe libraries are synthesized in such a way that the mass of each one of the components can be unequivocally assigned to its sequence.

The amplified products may also be influenced in another preferred variant of the method relative to their average size by modification of the time period of chain extension in the amplification step. In this case, since predominantly smaller fragments (approximately 200-500 base pairs) are investigated, a shortening of the chain extension steps, e.g., of a PCR, is meaningful.

In another preferred variant of the method, the amplified products are separated by gel electrophoresis, and the fragments in the desired size range are cut out prior to the analysis. In another particularly preferred variant, the amplified products that are cut out of the gel are again amplified with the use of the same set of primers. In this way, only fragments of the desired size can form, since others are no longer available as the template.

Another subject of the present invention is a kit containing at least two pairs of primers, reagents and adjuvants for the amplification and/or reagents and adjuvants for the chemical treatment and/or a combinatory probe library and/or an oligonucleotide array (DNA chip), as long as they are necessary or useful for conducting the method according to the invention.

The following examples explain the invention.

### EXAMPLES

### Example 1

### Primers for the Preferred Amplification of CG-Rich Regions in the Human Genome

CG-rich regions in the human genome are so-called CpG islands, which possess a regulatory function. We define CpG islands in such a way that they comprise at least 500 bp as well as have a GC content of >50%, and also the CG/GC quotient >0.6. Under these conditions, 16 Mb are present as CpG islands. Approximately 0.5% of the genomic sequence lies in these CpG islands, if one also considers a region of up to 1000 bp downstream each time. This consideration is based on data from the Ensembl Database of Oct. 31, 2000, Quelle Sanger Center. The sequence available therein comprised approximately 3.5 GB, and repeats were masked for the calculations.

It would be statistically expected for 12 mers that they hybridize only 0.005 time as frequently to one of the CG-rich regions than to another random region in the genome. Primers have now been found, which bind 1.8 times more frequently to a CG-rich region. Also, a specificity for these CpG islands results practically with the corresponding reverse primer that is found.

In this example, the primers are AGTAGTAGTAGT (Seq. ID 1), AAAACAAAAACC (Seq. ID 2) and alternatively AGTAGTAGTAGT (Seq. ID 19) and ACAAAAACTAAA (Seq. ID 20). The first pair of primers leads at least to the amplified products of Seq. ID 3 to 18, while the second pair of primers leads to the amplified products of Seq. ID 21 to 31.

### Example 2

### Calculation of the Predicted Number of Amplified Products in Genomic Regions

According to claim 8 of the patent, it is shown how to be able to prepare more than double the number of amplified products than would be statistically expected according to formula 1.

$$\begin{matrix}F = N * {P}_{s} ( Primers ) \frac{( {P}_{a} ( Primers ) )}{log ( 1 - {P}_{a} ( Primers ) )} \\ [ {( 1 - {P}_{a} ( Primers ) )}^{M} - 1 ] + \\ N * {P}_{a} ( Primers ) \frac{( {P}_{s} ( Primers ) )}{log ( 1 - {P}_{s} ( Primers ) )} \\ [ {( 1 - {P}_{s} ( Primers ) )}^{M} - 1 ]\end{matrix} \qquad Formula 1$$

F indicates the number of predicted amplified products, which are to be expected, if N bases are considered as the basis for the data from the genome. P is the respective probability for the hybridization of a primer oliogonucleotide, separated according to hybridization into the sense strand and the antisense strand. M is the maximal allowable length of the amplified products to be expected.

The probability P is determined by a Markov chain of the first order. The assumption is made that the DNA is a random sequence as a function of adjacent bases. For the calculation of a Markov chain, the transition probabilities of adjacent bases are necessary. These were empirically determined from 12% of the assembled human genome, which was completely treated with bisulfite and is compiled in Table 1. The transition probabilities for the corresponding complementary reverse strand are shown in Table 2. These result by simple permutation of the entries from Table 1.

TABLE 1

| From\to | A | C | G | T |
|---|---|---|---|---|
| A | 0.0894 | 0.0033 | 0.0722 | 0.1162 |
| C | 0.0 | 0.0 | 0.0140 | 0.0 |
| G | 0.0603 | 0.0036 | 0.0601 | 0.0959 |
| T | 0.1314 | 0.0071 | 0.0736 | 0.2729 |

with

P_{bDNA}(A)=0.2811

P_{bDNA}(C)=0.0140

P_{bDNA}(G)=0.2199

P_{bDNA}(T)=0.4850

and for the reverse complementary strand thereto (by corresponding exchange of the entires) P_{rbDNA} (from; to)

TABLE 2

| From\to | A | C | G | T |
|---|---|---|---|---|
| A | 0.2729 | 0.0959 | 0.0 | 0.1162 |
| C | 0.0736 | 0.0601 | 0.0140 | 0.0722 |
| G | 0.0071 | 0.0036 | 0.0 | 0.0033 |
| T | 0.1314 | 0.0603 | 0.0 | 0.0894 |

with

P_{rbDNA}(A)=0.4850

P_{rbDNA}(C)=0.2199

P_{rbDNA}(G)=0.0140

P_{rbDNA}(T)=0.2811

Thus the probability that a perfect base pairing results for a Primer PrimE (with the base sequence B₁B₂B₃B₄ . . . ; e.g., ATTG . . . ) depends on the precise sequence of bases and results as the product:

$${P}_{3 s} ( PrimE ) = {P}_{rbDNA} ( {B}_{1} ) \frac{{P}_{rbDNA} ( {B}_{1} ; {B}_{2} )}{{P}_{rbDNA} ( {B}_{1} )} \frac{{P}_{rbDNA} ( {B}_{2} ; {B}_{3} )}{{P}_{rbDNA} ( {B}_{2} )} \frac{{P}_{rbDNA} ( {B}_{3} ; {B}_{4} )}{{P}_{rbDNA} ( {B}_{3} )} …$$

(bisulfite DNA strand)

$${P}_{3 u} ( PrimE ) = {P}_{bDNA} ( {B}_{1} ) \frac{{P}_{bDNA} ( {B}_{1} ; {B}_{2} )}{{P}_{bDNA} ( {B}_{1} )} \frac{{P}_{bDNA} ( {B}_{2} ; {B}_{3} )}{{P}_{bDNA} ( {B}_{2} )} \frac{{P}_{bDNA} ( {B}_{3} ; {B}_{4} )}{{P}_{bDNA} ( {B}_{3} )} …$$

(anti-sense strand to a bisulfite DNA strand);

for a primer Prim, the number of perfect base pairings on the sense strand is

N*Ps(Prim)

If several primers (PrimU, PrimV, PrimW, Prim X, etc.) are used simultaneously, the following results as the probability for a perfect base pairing on the sense strand at a given position:

$$\begin{matrix}{P}_{s} ( Primers ) = {P}_{s} ( PrimU ) + \\ [ ( 1 - {P}_{s} ( PrimU ) ) {P}_{s} ( PrimV ) + \\ ( 1 - {P}_{s} ( PrimU ) ) ( 1 - {P}_{s} ( PrimV ) ) {P}_{s} ( PrimW ) + \\ ( 1 - {P}_{s} ( PrimU ) ) ( 1 - {P}_{s} ( PrimV ) ) ( 1 - {P}_{s} ( PrimW ) ) {P}_{s} ( PrimX )\end{matrix}$$

(PrimU, PrimV, Prim W . . . are different primers here with different base pairings). and thus the following is the number of perfect base pairings to be expected with any of the primers.

N*Pₛ(Primers).

Analogous equations are used for the determination of Pₐ (Primers) on the anti-sense strand.

For the example with two primers (a sense primer and an antisense primer), the following probabilities result:

P(CTAGTAGTAGT)=0.000000860027

P(AACAAAACTAA)=0.000030005828

The frequency of hybridizations to be expected on the CpG islands, which contain overall approximately 30,000,000 bases, is:

AGTAGTAGTAGT: 25.80 on the sense strand

AACAAAAACTAA: 900.17 on the complementary reverse stand.

The primers cannot be hybridized on the other strands each time, since Cs do not occur outside the context CG on the sense strand due to the bisulfite treatment and are thus correspondingly complementary to the anti-sense strand.

An amplified product is formed precisely if, in the case of a perfect base pairing on the sense strand, within the maximum fragment length M, a primer forms a perfect base pairing on the counterstrand; the probability for this is:

$${P}_{a} ( Primers ) {\sum }_{i = 0}^{M - 2} {( 1 - {P}_{a} ( Primers ) )}^{l} ;$$

For large M and small Pₐ (Primers) this is calculated by the following expression:

$$\frac{{P}_{a} ( Primers )}{log ( 1 - {P}_{a} ( Primers ) )} [ {( 1 - {P}_{a} ( Primers ) )}^{M} - 1 ] ;$$

The total number F of the amplified products, which are to be expected by the amplification of both strands, is thus:

$$\begin{matrix}F = N * {P}_{s} ( Primers ) \frac{( {P}_{a} ( Primers ) )}{log ( 1 - {P}_{a} ( Primers ) )} \\ [ {( 1 - {P}_{a} ( Primers ) )}^{M} - 1 ] + \\ N * {P}_{a} ( Primers ) \frac{( {P}_{s} ( Primers ) )}{log ( 1 - {P}_{s} ( Primers ) )} \\ [ {( 1 - {P}_{s} ( Primers ) )}^{M} - 1 ]\end{matrix} \qquad Formula 1$$

For the above-given example, 3.0498 amplified products result for the CpG islands with 30 megabases. We can show, however (see Example 1) that more than the statistically predicted amplifed products can be produced with primers that are specific for specific regions.

1. A method for the parallel detection of the methylation state of genomic DNA, hereby characterized in that the following steps are conducted:
a) in a genomic DNA sample, unmethylated cytosine bases at the 5′ position are converted by chemical treatment to uracil, thymidine or another base dissimilar to cytosine in its hybridization behavior;
b) more than ten different fragments, each of which is less than 2000 base pairs long, from this chemically treated genomic DNA are amplified simultaneously by use of synthetic oligonucleotides as primers, whereby each of these primers contains sequences of transcribed and/or translated genomic sequences and/or sequences that participate in gene regulation, as would be present after treatment according to step a);
c) the sequence context of all or part of the CpG dinucleotides or CpNpG trinucleotides contained in the amplified fragments is determined.

2. The method according to claim 1, further characterized in that the chemical treatment is conducted by means of a solution of a bisulfite, hydrogen sulfite or disulfite.

3. The method according to claim 1 or 2, further characterized in that at least one of the oligonucleotides used in step b) contains fewer nucleobases than would be necessary statistically for a sequence-specific hybridization to the chemically treated genomic DNA sample.

4. The method according to one of claims 1 to 3, further characterized in that at least one of the oligonucleotides used in step b) of claim 1 is shorter than 18 nucleobases.

5. The method according to one of claims 1 to 3, further characterized in that at least one of the oligonucleotides used in step b) of claim 1 is shorter than 15 nucleobases.

6. The method according to claim 1 or 2, further characterized in that more than 4 different oligonucleotides are used simultaneously for the amplification in step b) of claim 1.

7. The method according to claim 1 or 2, further characterized in that more than 26 different oligonucleotides are used simultaneously in step b) of claim 1 for the amplification.

8. The method according to one of the preceding claims, further characterized in that in step b) of claim 1, more than double the [number of] amplified fragments than calculated according to formula 1 originates from genomic segments, such as promoters and enhancers, that participate in the regulation of genes than would be expected in a purely random selection of oligonucleotide sequences, or their fraction of total detectable fragments is more than double that calculated according to formula 1,
$$\begin{matrix}F = N * {P}_{s} ( Primers ) \frac{( {P}_{a} ( Primers ) )}{log ( 1 - {P}_{a} ( Primers ) )} \\ [ {( 1 - {P}_{a} ( Primers ) )}^{M} - 1 ] + \\ N * {P}_{a} ( Primers ) \frac{( {P}_{s} ( Primers ) )}{log ( 1 - {P}_{s} ( Primers ) )} \\ [ {( 1 - {P}_{s} ( Primers ) )}^{M} - 1 ]\end{matrix} \qquad Formula 1$$
wherein the calculation is conducted as follows:
in the DNA treated with bisulfite, C can occur only in the context CG, so it is assumed that the primary DNA is a random sequence with dependence of directly adjacent bases (Markov chain of the first order); the base pairing probabilities determined empirically from the database (completely methylated; treated with bisulfite) are the same for both DNA strands as P_{bDNA} (from; to) from the following table:
TABLE 1

| From\to | A | C | G | T |
|---|---|---|---|---|
| A | 0.0894 | 0.0033 | 0.0722 | 0.1162 |
| C | 0.0 | 0.0 | 0.0140 | 0.0 |
| G | 0.0603 | 0.0036 | 0.0601 | 0.0959 |
| T | 0.1314 | 0.0071 | 0.0736 | 0.2729 |

with
P_{bDNA}(A)=0.2811
P_{bDNA}(C)=0.0140
P_{bDNA}(G)=0.2199
P_{bDNA}(T)=0.4850
and for the reverse-complementary strand thereto (by corresponding exchange of the entries) P_{rBDNA} (from;to)

| From\to | A | C | G | T |
|---|---|---|---|---|
| A | 0.2729 | 0.0959 | 0.0 | 0.1162 |
| C | 0.0736 | 0.0601 | 0.0140 | 0.0722 |
| G | 0.0071 | 0.0036 | 0.0 | 0.0033 |
| T | 0.1314 | 0.0603 | 0.0 | 0.0894 |

with
P_{rbDNA}(A)=0.4850
P_{rbDNA}(C)=0.2199
P_{rbDNA}(G)=0.0140
P_{rbDNA}(T)=0.2811
thus the probability that a perfect base pairing results for a primer PrimE (with the base sequence B₁B₂B₃B₄ . . . ; e.g. ATTG . . . ) depends on the precise sequence of the bases and results as the product:
$${P}_{3 s} ( PrimE ) = {P}_{rbDNA} ( {B}_{1} ) \frac{{P}_{rbDNA} ( {B}_{1} ; {B}_{2} )}{{P}_{rbDNA} ( {B}_{1} )} \frac{{P}_{rbDNA} ( {B}_{2} ; {B}_{3} )}{{P}_{rbDNA} ( {B}_{2} )} \frac{{P}_{rbDNA} ( {B}_{3} ; {B}_{4} )}{{P}_{rbDNA} ( {B}_{3} )} …$$
(bisulfite DNA strand)
$${P}_{3 u} ( PrimE ) = {P}_{bDNA} ( {B}_{1} ) \frac{{P}_{bDNA} ( {B}_{1} ; {B}_{2} )}{{P}_{bDNA} ( {B}_{1} )} \frac{{P}_{bDNA} ( {B}_{2} ; {B}_{3} )}{{P}_{bDNA} ( {B}_{2} )} \frac{{P}_{bDNA} ( {B}_{3} ; {B}_{4} )}{{P}_{bDNA} ( {B}_{3} )} …$$
(anti-sense strand to a bisulfite DNA strand);
[the number of] perfect base pairings for a primer Prim on the sense strand is
N*Pₛ(Prim);
If several primers (PrimU, PrimV, PrimW, PrimX, etc.) are used simultaneously, the probability for a perfect base pairing on the sense strand at a given position is:
$$\begin{matrix}{P}_{s} ( Primers ) = {P}_{s} ( PrimU ) + \\ ( 1 - {P}_{s} ( PrimU ) ) {P}_{s} ( PrimV ) + \\ ( 1 - {P}_{s} ( PrimU ) ) ( 1 - {P}_{s} ( PrimV ) ) {P}_{s} ( PrimW ) + \\ ( 1 - {P}_{s} ( PrimU ) ) ( 1 - {P}_{s} ( PrimV ) ) ( 1 - {P}_{s} ( PrimW ) ) {P}_{s} ( PrimX ) +\end{matrix}$$
and thus the number of perfect base pairings to be expected with any of the primers is:
N*Pₛ(Primers);
analogous equations are used for the determination of Pa (Primers) on the anti-sense strand; an amplified product is formed precisely if, in the case of a perfect base pairing on the sense strand, within the maximum fragment length M, a primer forms a perfect base pairing on the counterstrand; the probability for this is:
$${P}_{a} ( Primers ) {\sum }_{i = 0}^{M - 2} {( 1 - {P}_{a} ( Primers ) )}^{l} ;$$
for large M and small Pₐ (Primers), this is calculated by the following expression:
$$\frac{{P}_{a} ( Primers )}{log ( 1 - {P}_{a} ( Primers ) )} [ {( 1 - {P}_{a} ( Primers ) )}^{M} - 1 ] ;$$
for the total number F of amplified products, which are to be expected due to the amplification of the two strands, the following results:
$$\begin{matrix}F = N * {P}_{s} ( Primers ) \frac{( {P}_{a} ( Primers ) )}{log ( 1 - {P}_{a} ( Primers ) )} \\ [ {( 1 - {P}_{a} ( Primers ) )}^{M} - 1 ] + \\ N * {P}_{a} ( Primers ) \frac{( {P}_{s} ( Primers ) )}{log ( 1 - {P}_{s} ( Primers ) )} \\ [ {( 1 - {P}_{s} ( Primers ) )}^{M} - 1 ]\end{matrix} \qquad Formula 1$$

9. The method according to one of claims 1 to 7, further characterized in that in step b) of claim 1, more than double the number of amplified fragments than calculated according to claim 8 originates from the genomic segments, which are transcribed into mRNA in at least one cell of the respective organism, than would be expected in a purely random selection of oligonucleotide sequences, or their fraction of total detectable fragments is more than double that calculated according to claim 8.

10. The method according to one of claims 1 to 7, further characterized in that in step b) of claim 1, more than double the number of amplified fragments than calculated according to claim 8 originates from spliced genomic segments (exons) after transcription into mRNA than would be expected in a purely random selection of oligonucleotide sequences, or their fraction of total detectable fragments is more than double that calculated according to claim 8.

11. The method according to one of claims 1 to 7, further characterized in that in step b) of claim 1, more than double the number of amplified fragments than calculated according to claim 8 originate from genomic segments, which code for parts of one or more gene families, than would be expected in a purely random selection of oligonucleotide sequences, or their fraction of total detectable fragments is more than double that calculated according to claim 8.

12. The method according to one of claims 1 to 7, further characterized in that in step b) of claim 1, more than twice as many amplified fragments than calculated according to claim 8 originate from genomic segments, which contain sequences characteristic of so-called “matrix attachment sites” (MARs) than would be expected in a purely random selection of oligonucleotide sequences, or their fraction of total detectable fragments is more than double that calculated according to claim 8.

13. The method according to one of claims 1 to 7, further characterized in that in step b) of claim 1, more than double the number of amplified fragments than that calculated according to claim 8 originate from genomic segments, which organize the packing density of chromatin as so-called “boundary elements” than would be expected in a purely random selection of oligonucleotide sequences, or their fraction of total detectable fragments is more than double that calculated according to claim 8.

14. The method according to one of claims 1 to 7, further characterized in that in step b) of claim 1 more than double the number of amplified fragments than that calculated according to claim 8 originate from “multiple drug resistance gene” (MDR) promoters or coding regions than would be expected in a purely random selection of oligonucleotide sequences, or their fraction of total detectable fragments is more than double that calculated according to claim 8.

15. The method according to one of the preceding claims, further characterized in that for the amplification of the fragments described in claim 1, two oligonucleotides or two classes of oligonucleotides are used, one of which or one class of which can contain the base C, but not the base G, except in the context CpG or CpNpG, and the other of which or the other class of which can contain the base G, but not the base C, except in the context CpG or CpNpG.

16. The method according to one of claims 1 to 4, further characterized in that the amplification described in claim 1 is conducted by means of two oligonucleotides, one of which contains a sequence that is four to sixteen bases long, which is complementary or corresponds to a DNA that would be formed, if a DNA fragment of the same length to which one of the following transcription factors binds:

| | |
|---|---|
| AhR/Arnt | aryl hydrocarbon receptor/aryl hydro- |
| | carbon receptor nuclear translocator |
| Arnt | aryl hydrocarbon receptor nuclear |
| | translocator |
| AML-1a | CBFA2; core-binding factor, runt domain, |
| | alpha subunit 2 (acute myeloid leukemia |
| | 1; aml1 oncogene) |
| AP-1 | activator protein-1 (AP-1); Synonyme: |
| | c-Jun |
| C/EBP | CCAAT/enhancer binding protein |
| C/EBPalpha | CCAAT/enhancer binding protein (C/EBP), |
| | alpha |
| C/EBPbeta | CCAAT/enhancer binding protein (C/EBP), |
| | beta |
| CDP | CUTL1; cut (Drosophila)-like 1 (CCAAT |
| | displacement protein) |
| CDP | CUTL1; cut (Drosophila)-like 1 (CCAAT |
| | displacement protein) |
| CDP CR1 | complement component (3b/4b) receptor 1 |
| CDP CR3 | complement component (3b/4b) receptor 3 |
| CHOP-C/ | DDIT; DNA-damage-inducible transcript |
| EBPalpha | 3/CCAAT/enhancer binding protein |
| | (C/EBP), alpha |
| c-Myc/Max | avian myelocytomatosis viral oncogene/ |
| | MYC-ASSOCIATED FACTOR X |
| CREB | cAMP responsive element binding protein |
| CRE-BP1 | CYCLIC AMP RESPONSE ELEMENT-BINDING |
| | PROTEIN 2, CREB2, CREBP1; now ATF2; |
| | activating transcription factor 2 |
| CRE-BP1/ | activator protein-1 (AP-1); Synonyme: |
| c-Jun | c-Jun |
| CREB | MP responsive element binding protein |
| E2F | E2F transcription factor (originally |
| | identified as a DNA-binding protein |
| | essential E1A-dependent activation of |
| | the adenovirus E2 promoter) |
| E47 | transcription factor 3 (E2A immuno- |
| | globulin enhancer binding factors |
| | E12/E47) |
| E47 | transcription factor 3 (E2A immuno- |
| | globulin enhancer binding factors |
| | E12/E47) |
| Egr-1 | early growth response 1 |
| Egr-2 | early growth response 2 (Krox-20 |
| | (Drosophila) homolog) |
| ELK-1 | ELK1, member of ETS (environmental |
| | tobacco smoke) oncogene family |
| Freac-2 | FKHL6; forkhead (Drosophila)-like 6; |
| | FORKHEAD-RELATED ACTIVATOR 2; FREAC2 |
| Freac-3 | FKHL7; forkhead (Drosophila)-like 7; |
| | FORKHEAD-RELATED ACTIVATOR 3; FREAC3 |
| Freac-4 | FKHL8; forkhead (Drosophila)-like 8; |
| | FORKHEAD-RELATED ACTIVATOR 4; FREAC4 |
| Freac-7 | FKHL11; forkhead (Drosophila)-like 9; |
| | FORKHEAD-RELATED ACTIVATOR 7; FREAC7 |
| GATA-1 | GATA-binding protein 1/Enhancer-Binding |
| | Protein GATA1 |
| GATA-1 | GATA-binding protein 1/Enhancer-Binding |
| | Protein GATA1 |
| GATA-1 | GATA-binding protein 1/Enhancer-Binding |
| | Protein GATA1 |
| GATA-2 | GATA-binding protein 2/Enhancer-Binding |
| | Protein GATA2 |
| GATA-3 | GATA-binding protein 3/Enhancer-Binding |
| | Protein GATA3 |
| GATA-X | |
| HFH-3 | FKHL10; forkhead (Drosophila)-like 10; |
| | FORKHEAD-RELATED ACTIVATOR 6; FREAC6 |
| HNF-1 | TCF1; transcription factor 1, hepatic; |
| | LF-B1, hepatic nuclear factor (HNF1), |
| | albumin proximal factor |
| HNF-4 | hepatocyte nuclear factor 4 |
| IRF-1 | interferon regulatory factor 1 |
| ISRE | interferon-stimulated response element |
| Lmo2 | LIM domain only 2 (rhombotin-like 1) |
| complex | |
| MEF-2 | MADS box transcription enhancer factor |
| | 2, polypeptide A (myocyte enhancer |
| | factor 2A) |
| MEF-2 | MADS box transcription enhancer factor |
| | 2, polypeptide A (myocyte enhancer |
| | factor 2A) |
| myogenin/ | Myogenin (myogenic factor 4)/Neuro- |
| NF-1 | fibromin 1; NEUROFIBROMATOSIS, TYPE I |
| MZF1 | ZNF42; zinc finger protein 42 |
| | (myeloid-specific retinoic acid- |
| | responsive) |
| MZF1 | ZNF42; zinc finger protein 42 |
| | (myeloid-specific retinoic acid- |
| | responsive) |
| NF-E2 | NFE2; nuclear factor (erythroid- |
| | derived 2), 45 kD |
| NF-kappaB | nuclear factor of kappa light poly- |
| (p50) | peptide gene enhancer in B-cells p50 |
| | subunit |
| NF-kappaB | nuclear factor of kappa light poly- |
| (p65) | peptide gene enhancer in B-cells p65 |
| | subunit |
| NF-kappaB | nuclear factor of kappa light poly- |
| | peptide gene enhancer in B-cells |
| NF-kappaB | nuclear factor of kappa light poly- |
| | peptide gene enhancer in B-cells |
| NRSF | NEURON RESTRICTIVE SILENCER FACTOR; |
| | REST; RE1-silencing transcription |
| | factor |
| Oct-1 | OCTAMER-BINDING TRANSCRIPTION FACTOR 1; |
| | POU2F1; POU domain, class 2, |
| | transcription factor 1 |
| Oct-1 | OCTAMER-BINDING TRANSCRIPTION FACTOR 1; |
| | POU2F1; POU domain, class 2, |
| | transcription factor 1 |
| Oct-1 | OCTAMER-BINDING TRANSCRIPTION FACTOR 1; |
| | POU2F1; POU domain, class 2, |
| | transcription factor 1 |
| Oct-1 | OCTAMER-BINDING TRANSCRIPTION FACTOR 1; |
| | POU2F1; POU domain, class 2, |
| | transcription factor 1 |
| Oct-1 | OCTAMER-BINDING TRANSCRIPTION FACTOR 1; |
| | POU2F1; POU domain, class 2, |
| | transcription factor 1 |
| P300 | E1A (adenovirus E1A oncoprotein)- |
| | BINDING PROTEIN, 300-KD |
| P53 | tumor protein p53 (Li-Fraumeni |
| | syndrome); TP53 |
| Pax-1 | paired box gene 1 |
| Pax-3 | paired box gene 3 (Waardenburg |
| | syndrome 1) |
| Pax-6 | paired box gene 6 (aniridia, keratitis) |
| Pbx 1b | pre-B-cell leukemia transcription factor |
| Pbx-1 | pre-B-cell leukemia transcription factor 1 |
| RORalpha2 | RAR-RELATED ORPHAN RECEPTOR ALPHA; |
| | RETINOIC ACID-BINDING RECEPTOR ALPHA |
| RREB-1 | ras responsive element binding protein 1 |
| SP1 | simian-virus-40-protein-1 |
| SP1 | simian-virus-40-protein-1 |
| SREBP-1 | sterol regulatory element binding |
| | transcription factor 1 |
| SRF | serum response factor (c-fos serum |
| | response element-binding transcription |
| | factor) |
| SRY | sex determining region Y |
| STAT3 | signal transducer and activator of |
| | transcription 1, 91 kD |
| Tal-1al- | T-cell acute lymphocytic leukemia |
| pha/E47 | 1/transcription factor 3 (E2A immuno- |
| | globulin enhancer binding factors |
| | E12/E47) |
| TATA | cellular and viral TATA box elements |
| Tax/CREB | Transiently-expressed axonal glyco- |
| | protein/cAMP responsive element binding |
| | protein |
| Tax/CREB | Transiently-expressed axonal glyco- |
| | protein/cAMP responsive element binding |
| | protein |
| TCF11/MafG | v-maf musculoaponeurotic fibrosarcoma |
| | (avian) oncogene family, protein G |
| TCF11 | Transcription Factor 11; TCF11; NFE2L1; |
| | nuclear factor (erythroid-derived |
| | 2)-like 1 |
| USF | upstream stimulating factor |
| Whn | winged-helix nude |
| X-BP-1 | X-box binding protein 1 oder |
| YY1 | ubiquitously distributed transcription |
| | factor belonging to theGLI-Kruppel |
| | class of zinc finger proteins |

would be subjected to a chemical treatment according to claim 1.

17. The method according to one of claims 1 to 4, further characterized in that the amplification described in claim 1 is conducted by means of two oligonucleotides, one of which contains the sequence that is four to sixteen bases long, which is complementary or corresponds to a DNA that would be formed if a DNA fragment of the same length, which can bring about the specific localization of genome/chromatin segments within the cell nucleus by means of its sequence or secondary structure, would be subjected to a chemical treatment according to claim 1.

18. The method according to one of claims 1 to 4, further characterized in that the amplification described in claim 1 is conducted by means of two oligonucleotides, at least [one] of which contains one of the sequences (from 5′ to 3′)
TCGCGTGTA, TACACGCGA, TGTACGCGA, TCGCGTACA,
TTGCGTGTT, AACACGCAA, GGTACGTAA, TTACGTACC,
TCGCGTGTT, AACACGCGA, GGTACGCGA, TCGCGTACC,
TTGCGTGTA, TACACGCAA, TGTACGTAA, TTACGTACA,
TACGTG, CACGTA, TACGTG, CACGTA,
ATTGCGTGT, ACACGCAAT, GTACGTAAT, ATTACGTAC,
ATTGCGTGA, TCACGCAAT, TTACGTAAT, ATTACGTAA,
ATCGCGTGA, TCACGCGAT, TTACGCGAT, ATCGCGTAA,
ATCGCGTGT, ACACGCGAT, GTACGCGAT, ATCGCGTAC,
TGTGGT, ACCACA, ATTATA, TATAAT,
TGAGTTAG, CTAACTCA, TTGATTTA, TAAATCAA,
TGATTTAG, CTAAATCA, TTGAGTTA, TAACTCAA,
TTTGGT, ACCAAA, ATTAAA, TTTAAT,
TGTGGA, TCCACA, TTTATA, TATAAA,
TTTGGA, TCCAAA, TTTAAA, TTTAAA,
TGTGGT, ACCACA, ATTATA, TATAAT,
ATTAT, ATAAT, GTAAT, ATTAC,
ATTGT, ACAAT, GTAAT, ATTAC,
GAAAG, CTTTC, TTTTT, AAAAA,
GTAAT, ATTAC, ATTGT, ACAAT,
GAAAT, ATTTC, ATTTT, AAAAT,
GTAAG, CTTAC, TTTGT, ACAAA,
TTAATAATCGAT, ATCGATTATTAA, ATCGATTATTGG,
CCAATAATCGAT, ATCGATTA, TAATCGAT, TAATCGAT,
ATCGATTA,
ATCGATCGG, CCGATCGAT, TCGATCGAT, ATCGATCGA,
ATCGATCGT, ACGATCGAT, GCGATCGAT, ATCGATCGC,
TATCGATA, TATCGATA, TATCGGTG, CACCGATA,
TATTAATA, TATTAATA, TATTGGTG, CACCAATA,
GTGTAATATTT, AAATATTACAC, GGGTATTGTAT,
ATACAATACCC, GTGTAATTTTT, AAAAATTACAC,
GGGGATTGTAT, ATACAATCCCC, ATGTAATTTTT,
AAAAATTACAT, GGGGATTGTAT, ATACAATCCCC,
ATGTAATATTT, AAATATTACAT, GGGTATTGTAT,
ATACAATACCC, ATTACGTGGT, ACCACGTAAT,
ATTACGTGGT, ACCACGTAAT,
TGACGTAA, TTACGTCA, TTACGTTA, TAACGTAA,
TGACGTTA, TAACGTCA, TGACGTTA, TAACGTCA,
TTACGTAA, TTACGTAA, TTACGTAA, TTACGTAA,
TGACGTTA, TAACGTCA, TAACGTTA, TAACGTTA,
TGACGT, ACGTCA, GCGTTA, TAACGC,
TGACGT, ACGTCA, ACGTTA, TAACGT,
TTTCGCGT, ACGCGAAA, GCGCGAAA, TTTCGCGC,
TTTGGCGT, ACGCCAAA, GCGTTAAA, TTTAACGC,
TAGGTGTTA, TAACACCTA, TAATATTTG, CAAATATTA,
TAGGTGTTT, AAACACCTA, GAATATTTG, CAAATATTC,
GTAGGTGG, CCACCTAC, TTATTTGT, ACAAATAA,
GTAGGTGT, ACACCTAC, ATATTTGT, ACAAATAT,
TGCGTGGGCGG, CCGCCCACGCA, TCGTTTACGTA,
TACGTAAACGA, TGCGTGGGCGT, ACGCCCACGCA,
ACGTTTACGTA, TACGTAAACGT,
TGCGTAGGCGT, ACGCCTACGCA, ACGTTTACGTA,
TACGTAAACGT, TGCGTAGGCGG, CCGCCTACGCA,
TCGTTTACGTA, TACGTAAACGA, ATAGGAAGT, ACTTCCTAT,
ATTTTTTGT, ACAAAAAAT,
TCGGAAGT, ACTTCCGA, ATTTTCGG, CCGAAAAT,
TCGGAAGT, ACTTCCGA, GTTTTCGG, CCGAAAAC,
TCGGAAAT, ATTTCCGA, ATTTTCGG, CCGAAAAT,
TCGGAAAT, ATTTCCGA, GTTTTCGG, CCGAAAAC,
GTAAATAA, TTATTTAC, TTGTTTAT, ATAAACAA,
GTAAATAAATA, TATTTATTTAC, TGTTTATTTAT,
ATAAATAAACA,
AAAGTAAATA, TATTTACTTT, TGTTTATTTT, AAAATAAACA,
AATGTAAATA, TATTTACATT, TGTTTATATT, AATATAAACA,
TAAGTAAATA, TATTTACTTA, TGTTTATTTA, TAAATAAACA,
TATGTAAATA, TATTTACATA, TGTTTATATA, TATATAAACA,
ATAAATA, TATTTAT, TGTTTAT, ATAAACA,
ATAAATA, TATTTAT, TATTTAT, ATAAATA,
GATA, TATC, TATT, AATA,
TAGATAA, TTATCTA, TTATTTG, CAAATAA,
TTGATAA, TTATGAA, TTATTAG, CTAATAA,
GATAA, TTATC, TTATT, AATAA,
GATG, CATC, TATT, AATA,
GATAG, CTATC, TTATT, AATAA,
GATAAG, CTTATC, TTTATT, AATAAA,
TGTTTATTTA, TAAATAAACA, TAAATAAATA, TATTTATTTA,
TGTTTGTTTA, TAAACAAACA, TAAATAAATA, TATTTATTTA,
TATTTATTTA, TAAATAAATA, TAAATAAATA, TATTTATTTA,
TATTTGTTTA, TAAACAAATA, TAAATAAATA, TATTTATTTA,
GTTAATGATT, AATCATTAAC, AATTATTAAT, ATTAATAATT,
GTTAATTATT, AATAATTAAC, AATAATTAAT, ATTAATTATT,
GTTAATTAAT, ATTAATTAAC, ATTAATTAAT, ATTAATTAAT,
GTTAATGAAT, ATTCATTAAC, ATTTATTAAT, ATTAATAAAT,
TAAAGTTTA, TAAACTTTA, TGAATTTTG, CAAAATTCA,
TAAAGGTTA, TAACCTTTA, TGATTTTTG, CAAAAATCA,
AAAGTGAAATT, AATTTCACTTT, GGTTTTATTTT,
AAAATAAAACC, AAAGCGAAATT, AATTTCGCTTT,
GGTTTCGTTTT, AAAACGAAACC,
TAGTTTTATTTTTTT, AAAAAAATAAAACTA, GGGAAAGTGAAATTG,
CAATTTCACTTTCCC,
TAGTTTTATTTTTTT, AAAAAAATAAAACTA, GGAAAAGTGAAATTG,
CAATTTCACTTTTCC,
TAGTTTTTTTTTTTT, AAAAAAAAAAAACTA, GGAAAAGAGAAATTG,
CAATTTCTCTTTTCC,
TAGTTTTTTTTTTTT, AAAAAAAAAAAACTA, GGGAAAGAGAAATTG,
CAATTTCTCTTTCCC,
TAGGTG, CACCTA, TATTTG, CAAATA,
TTTTAAAAATAATTTT, AAAATTATTTTTAAAA,
AGGGTTATTTTTAGAG, CTCTAAAAATAACCCT,
TTTTAAAAATAATTTT, AAAATTATTTTTAAAA,
GGAGTTATTTTTAGAG, CTCTAAAAATAACTCC,
TTTTAAAAATAATTTT, AAAATTATTTTTAAAA,
AGAGTTATTTTTAGAG, CTCTAAAAATAACTCT,
TTTTAAAAATAATTTT, AAAATTATTTTTAAAA,
GGGGTTATTTTTAGAG, CTCTAAAAATAACCCC,
TGTTATTAAAAATAGAAA, TTTCTATTTTTAATAACA,
TTTTTATTTTTAGTAATA, TATTACTAAAAATAAAAA,
TGTTATTAAAAATAGAAT, ATTCTATTTTTAATAACA,
GTTTTATTTTTAGTAATA, TATTACTAAAAATAAAAC,
TTTGGTAT, ATACCAAA, GTGTTAAA, TTTAACAC
GGGGA, TCCCC, TTTTT, AAAAA,
TAGGGG, CCCCTA, TTTTTA, TAAAAA,
GAGGGG, CCCCTC, TTTTTT, AAAAAA,
TGTTGAGTTAT, ATAACTCAACA, ATGATTTAGTA,
TACTAAATCAT, TGTTGATTTAT, ATAAATCAACA,
GTGAGTTAGTA, TACTAACTCAC, TGTTGAGTTAT,
ATAACTCAACA, ATGATTTAGTA, TACTAAATCAT,
TGTTGATTTAT, ATAAATCAACA, GTGAGTTAGTA,
TACTAACTCAC,
GGGGATTTTT, AAAAATCCCC, GGGAATTTTT, AAAAATTCCC,
GGGGATTTTT, AAAAATCCCC, GGGGATTTTT, AAAAATCCCC,
GGGGATTTTT, AAAAATCCCC, GGAAATTTTT, AAAAATTTCC,
GGGAATTTTT, AAAAATTCCC, GGAAATTTTT, AAAAATTTCC,
GGGAATTTTT, AAAAATTCCC, GGAAATTTTT, AAAAATTTCC,
GGGATTTTTT, AAAAAATCCC, GGAAAGTTTT, AAAACTTTCC,
GGGAATTTTT, AAAAATTCCC, GGGAATTTTT, AAAAATTCCC,
GGGATTTTTT, AAAAAATCCC, GGGAAGTTTT, AAAACTTCCC,
GGGATTTTTTA, TAAAAAATCCC, TGGAAAGTTTT,
AAAACTTTCCA, TTTAGTATTACGGATAGAGGT,
ACCTCTATCCGTAATACTAAA, GTTTTTGTTCGTGGTGTTGAA,
TTCAACACCACGAACAAAAAC, TTTAGTATTACGGATAGAGTT,
AACTCTATCCGTAATACTAAA, GGTTTTGTTCGTGGTGTTGAA,
TTCAACACCACGAACAAAACC, TTTAGTATTACGGATAGCGTT,
AACGCTATCCGTAATACTAAA, GGCGTTGTTCGTGGTGTTGAA,
TTCAACACCACGAACAACGCC, TTTAGTATTACGGATAGCGGT,
ACCGCTATCCGTAATACTAAA, GTCGTTGTTCGTGGTGTTGAA,
TTCAACACCACGAACAACGAC,
ATATGTAAAT, ATTTACATAT, ATTTGTATAT, ATATACAAAT,
TTATGTAAAT, ATTTACATAA, ATTTGTATAA, TTATACAAAT,
GAATATTTA, TAAATATTC, TGAATATTT, AAATATTCA,
GAATATGTA, TACATATTC, TGTATATTT, AAATATACA,
ATAAT, ATTAT, ATTAT, ATAAT,
GTAAT, ATTAC, ATTAT, ATAAT,
AATGTAAAT, ATTTACATT, ATTTGTATT, AATACAAAT,
ATTTGTATATT, AATATACAAAT, GGTATGTAAAT, ATTTACATACC,
ATTTGTATATT, AATATACAAAT, AATATGTAAAT, ATTTACATATT,
ATTTGTATATT, AATATACAAAT, AGTATGTAAAT, ATTTACATACT,
ATTTGTATATT, AATATACAAAT, GATATGTAAAT, ATTTACATATC,
AGGAGT, ACTCCT, ATTTTT, AAAAAT,
GGGAGT, ACTCCC, ATTTTT, AAAAAT,
GGATATGTTCGGGTATGTTT, AAACATACCCGAACATATCC,
GGATATGTTCGGGTATGTTT, AAACATACCCGAACATATCC,
GGATATGTTCGGGTATGTTT, AAACATACCCGAACATATCC,
AGATATGTTCGGGTATGTTT, AAACATACCCGAACATATCT,
TCGTTTCGTTTTAGATAT, ATATCTAAAACGAAACGA,
ATATTTAGAGCGGAACGG, CCGTTCCGCTCTAAATAT,
CGTTACGGTT, AACCGTAACG, AATCGTGACG, CGTCACGATT,
CGTTACGGTT, AACCGTAACG, GATCGTGACG, CGTCACGATC,
CGTTACGTTT, AAACGTAACG, AAGCGTGACG, CGTCACGCTT,
CGTTACGTTT, AAACGTAACG, GAGCGTGACG, CGTCACGCTC,
TTTACGTATGA, TCATACGTAAA, TTATGCGTGAA,
TTCACGCATAA, TTTACGTTTGA, TCAAACGTAAA,
TTAAGCGTGAA, TTCACGGTTAA, TTTACGTTTTA,
TAAAACGTAAA, TGAAGCGTGAA, TTCACGCTTCA,
TTTACGTATTA, TAATACGTAAA, TGATGCGTGAA,
TTCACGCATCA,
AATTAATTAA, TTAATTAATT, TTGATTGATT, AATCAATCAA,
TATTAATTAA, TTAATTAATA, TTGATTGATG, CATCAATCAA,
TAATTAT, ATAATTA, ATGATTG, CAATCAT,
TAGGTTA, TAACCTA, TGATTTA, TAAATCA,
TTTTAAATATTTTT, AAAAATATTTAAAA, GGGGGTGTTTGGGG,
CCCCAAACACCCCC,
TTTTAAATTATTTT, AAAATAATTTAAAA, GGGGTGGTTTGGGG,
CCCCAAACCACCCC,
TTTTAAATTTTTTT, AAAAAAATTTAAAA, GGGGGGGTTTGGGG,
CCCCAAACCCCCCC,
TTTTAAATAATTTT, AAAATTATTTAAAA, GGGGTTGTTTGGGG,
CCCCAAACAACCCC,
GAGGCGGGG, CCCCGCCTC, TTTCGTTTT, AAAACGAAA,
GAGGTAGGG, CCCTACCTC, TTTTGTTTT, AAAACAAAA,
AAGGCGGGG, CCCCGCCTT, TTTCGTTTT, AAAACGAAA,
AAGGTAGGG, CCCTACCTT, TTTTGTTTT, AAAACAAAA,
GGGGGCGGGGT, ACCCCGCCCCC, ATTTCGTTTTT,
AAAAACGAAAT, GGGGGCGGGGT, ACCCCGCCCCC,
GTTTCGTTTTT, AAAAACGAAAC, TATTATTTTAT,
ATAAAATAATA, GTGGGGTGATA, TATCACCCCAC,
GATTATTTTAT, ATAAAATAATC, GTGGGGTGATT,
AATCACCCCAC,
ATTACGTGAT, ATCACGTAAT, ATTACGTGAT, ATCACGTAAT,
ATTACGTGAT, ATCACGTAAT, GTTACGTGAT, ATCACGTAAC,
TTTTATATGG, CCATATAAAA, TTATATAAGG, CCTTATATAA,
TTATATATGG, CCATATATAA, TTATATATGG, CCATATATAA,
AAATAAT, ATTATTT, GTTGTTT, AAACAAC,
AAATTAA, TTAATTT, TTAGTTT, AAACTAA,
AAATTAT, ATAATTT, GTAGTTT, AAACTAC,
AAATAAA, TTTATTT, TTTGTTT, AAACAAA,
ATTTTTCGGAAATG, CATTTCCGAAAAAT, TATTTTCGGGAAAT,
ATTTCCCGAAAATA,
ATTTTTCGGAAATG, CATTTCCGAAAAAT, TATTTTCGGGAAAT,
ATTTCCCGAAAATA,
ATTTTCGGGAAATG, CATTTCCCGAAAAT, TATTTTTCGGAAAT,
ATTTCCGAAAAATA,
ATTTTCGGGAAGTG, CACTTCCCGAAAAT, TATTTTTCGGAAAT,
ATTTCCGAAAAATA,
AATAGATGTT, AACATCTATT, AATATTTGTT, AACAAATATT,
AATAGATGGT, ACCATCTATT, ATTATTTGTT, AACAAATAAT,
GTATAAATA, TATTTATAC, TATTTATAT, ATATAAATA,
GTATAAATG, CATTTATAC, TATTTATAT, ATATAAATA,
GTATAAAAA, TTTTTATAC, TTTTTATAT, ATATAAAAA,
GTATAAAAG, CTTTTATAC, TTTTTATAT, ATATAAAAA,
TTATAAATA, TATTTATAA, TATTTATAG, CTATAAATA,
TTATAAATG, CATTTATAA, TATTTATAG, CTATAAATA,
TTATAAAAA, TTTTTATAA, TTTTTATAG, CTATAAAAA,
TTATAAAAG, CTTTTATAA, TTTTTATAG, CTATAAAAA,
GGGGGTTGACGTA, TACGTCAACCCCC, TGCGTTAATTTTT,
AAAAATTAACGCA,
GGGGGTTGACGTA, TACGTCAACCCCC, TACGTTAATTTTT,
AAAAATTAACGTA,
TGACGTATATTTTT, AAAAATATACGTCA, GGGGATATGCGTTA,
TAACGCATATCCCC,
TGACGTATATTTTT, AAAAATATACGTCA, GGGGGTATGCGTTA,
TAACGCATACCCCC,
ATGATTTAGTA, TACTAAATCAT, TGTTGAGTTAT,
ATAACTCAACA, GTTAT, ATAAC, ATGAT, ATCAT,
TTACGTGA, TGACGTAA, TTACGTGG, CCACGTAA,
TTACGTGG, CCACGTAA, TTACGTGG, CCACGTAA,
TTACGTGG, CCACGTAA, TTACGTGA, TCACGTAA,
TTACGTGA, TCACGTAA, TTACGTGA, TCACGTAA,
GACGTT, AACGTC, AGCGTT, AACGCT,
TGACGTGT, ACACGTCA, ATACGTTA, TAACGTAT,
TGACGTGG, CCACGTCA, TTACGTTA, TAACGTAA,
CGGTTATTTTG, CAAAATAACCG, TAAGATGGTCG oder
CGACCATCTTA
which is complementary or corresponds to a DNA that would be formed if a DNA fragment of the same length, which can bring about the specific localization of genome/chromatin segments within the cell nucleus via its sequence or secondary structure, would be subjected to a chemical treatment according to claim 1.

19. The method according to one of claims 16 to 18, further characterized in that the oligonucleotides used for the amplification, outside the consensus sequences defined in claim 16 to 18, contain several positions at which either any of the three bases G, A and T or any of the three bases C, A and T can be present.

20. The method according to claim 19, further characterized in that the oligonucleotides used for the amplification, outside of one of the consensus sequences described in claim 18, contain only as many additional bases as is necessary for the simultaneous amplification of more than one hundred different fragments per reaction of chemically treated DNA, calculated according to claim 8.

21. The method according to one of the preceding claims, further characterized in that the investigation of the sequence context of all or part of the CpG dinucleotides or CpNpGp trinucleotides contained in the amplified fragments undertaken according to claim 1c) is conducted by hybridizing the fragments already provided with a fluorescence marker in the amplification to an oligonucleotide array (DNA chip).

22. The method according to one of claims 1 to 20, further characterized in that the amplified fragments [are] immobilized on a surface and then a hybridization is conducted with a combinatory library of distinguishable oligonucleotide or PNA oligomer probes.

23. The method according to claim 22, further characterized in that the probes are detected based on their unequivocal mass by means of matrix-assisted laser desorption/ionization mass spectrometry (MALDI-MS), and thus the sequence context of all or a part of the CpG dinucleotides or CpNpGp trinucleotides contained in the amplified fragments is decoded.

24. The method according to one of the preceding claims, further characterized in that the amplification is conducted as described in step b) of claim 1 by a polymerase chain reaction, in which the size of the amplified fragments is limited by means of chain extension steps that are shortened to less than 30 s.

25. The method according to one of the preceding claims, further characterized in that after the amplification according to step b) of claim 1, the products are separated by gel eletrophoresis and the fragments, which are smaller than 2000 base pairs or smaller than a random limiting value below 2000 base pairs, are separated by cutting them out from the other products of the amplification prior to the evaluation according to step c) of claim 1.

26. The method according to claim 25, further characterized in that after the separation of amplified products of specific size, these products are amplified once more prior to conducting step c) of claim 1.

27. A kit, containing at least two pairs of primers, reagents and adjuvants for the amplification and/or reagents and adjuvants for the chemical treatment according to claim 1a) and/or a combinatory probe library and/or an oligonucleotide array (DNA chip) as long as they are necessary or useful for conducting the method according to the invention.
